(19) United States
(12) Patent Application Publication
(10) Pub. No.: US 2026/0034135 A1
(43) Pub. Date: Feb. 5, 2026

(54) USE OF N6-BENZYLAMINOPURINE AS AN APPETITE SUPPRESSANT

(71) Applicant: THE GOVERNING COUNCIL OF THE UNIVERSITY OF TORONTO, TORONTO (CA)

(72) Inventor: DENISE DORIS BELSHAM, TORONTO (CA)

(21) Appl. No.: 19/099,496

(22) PCT Filed: Jul. 26, 2023
(86) PCT No.: PCT/CA2023/051000

§ 371 Date: Jan. 29, 2025

Related U.S. Application Data
(60) Provisional application No. 63/393,528, filed on Jul. 29, 2022.

(51) Int. Cl. A61K 31/52; A61P 3/04

(52) U.S. Cl. CPC A61K 31/52; A61P 3/04

(57) ABSTRACT

Disclosed herein is the use of N6-benzylaminopurine (BAP) as an appetite suppressant in animals including humans. The results of studies demonstrate the use of BAP as an appetite suppressant in animals, and the use of this common agricultural supplement for plant growth can be used to help to alleviate the ever-growing obesity pandemic afflicting modern society.

### FIELD

The present disclosure relates to the use of N6-benzylaminopurine (BAP) as an appetite suppressant in animals including humans.

### BACKGROUND

Obesity is a global health pandemic with the World Health Organization estimating that nearly 2 billion people worldwide are obese or overweight. Obesity is accompanied by dangerous comorbidities such as type 2 diabetes mellitus, cardiovascular disease, cancer, and depression. This pandemic has been estimated to cost billions in healthcare costs and lost productivity. Despite the ever-growing size of this pandemic, there is a glaring lack of effective pharmaceutical treatment options for obesity. As only 1-2% of obese patients receive pharmaceutical intervention, which are often costly and can only achieve modest reductions in body weight (1, 2). The primary cause of obesity is positive energy balance, wherein people consume more energy than they expend. A potential future obesity therapeutic is the cytokinin, N6-benzylaminopurine (BAP), see https://pubchem.ncbi.nlm.nih.gov/compound/6-Benzylaminopurine.

The hypothalamus is a small region of the brain that controls numerous critical physiological functions to maintain homeostasis, including feeding behavior, reproduction, and stress. In order to control these diverse functions, the hypothalamus contains distinct neuronal subpopulations. It maintains energy balance via two distinct opposing neuronal subpopulations in the arcuate nucleus, orexigenic, appetite-inducing, neuropeptide Y (NPY)/agouti-related peptide (AgRP), and anorexigenic, appetite-supressing, proopiomelanocortin (POMC)-expressing neurons (3). Disruptions to the homeostatic biology of these neurons can lead to weight gain and predispose one to obesity. In the obese state, these neurons are drastically altered, as the neurons become resistant to peripheral satiety signals, such as insulin and leptin (4, 5). Mechanistic studies in specific neuropeptide expressing neurons are difficult due to the heterogeneous nature of this brain region. To circumvent this complexity, our lab has generated an array of clonal immortalized hypothalamic neurons, each with their own unique expression profiles, to precisely study effects and mechanisms in specific hypothalamic neuronal subpopulations (6).

The cytokinins are a class of plant hormones that promote cell division and direct differentiation. They have been widely used in the agriculture industry to promote plant growth and flowering. In mammalian cells, cytokinins have been reported to have antioxidant and anti-aging properties (7, 8). There are two major classes of cytokinins, adenine-and phenyl urea-derived. Adenine-derived cytokinins, including kinetin, zeatin, and BAP, have been reported to act on mammalian cells via the P2 family of purine receptors, which endogenously bind a variety of purine derivatives, including ATP, ADP, and UDP (9). Our candidate cytokinin, BAP, is an adenine-derived cytokinin with a benzyl group substituted at N6. The literature surrounding BAP is limited to a handful of papers. In rats, BAP was shown to act via P2 receptors to increase the strength of atrial muscle contractions by modifying intracellular calcium levels and cGMP (10). In murine derived B16 melanoma cells, BAP was able to induce melanogenesis in a protein kinase A-dependent manner (11). A previous study conducted by the environmental protection agency (EPA-see attached) reported that rats fed BAP for 13 weeks had reduced weight gain and food intake, demonstrating the potential weight reducing effects of BAP, although no data was shown to support this statement.

### SUMMARY

Herein we report the effects of BAP on body weight in CD-1 mice. In CD-1 mice, BAP reduces body weight in male mice via two different oral administration methods, in the water or in a food-based emulsion, while on a 60% high fat diet (HFD) or sucrose-matched control diet; whereas it is only able to reduce body weight in female CD-1 mice that are on a 60% HFD (not on a regular chow diet). We find that in both male and female mice fed a 60% HFD, the mice exposed to BAP lose weight, maintain their weight and/or do not continue to gain weight at the same rate as the control mice. Furthermore, we report the effects of BAP on gene expression across multiple hypothalamic neuronal cell lines and in primary hypothalamic culture. We also explore mechanisms by which BAP may act in neurons through purine receptors and other cellular signal transduction pathways.

Thus, the present disclosure provides an appetite suppressant administered to a subject comprising N6-benzylaminopurine and derivatives thereof formulated in an administrable form to the subject.

The N6-benzylaminopurine may be present in the administrable form in a concentration range from about 50 to about 2000 milligrams/kilogram of the subject's weight.

The N6-benzylaminopurine may be present in the administrable form in a concentration range from about 100 to about 500 milligrams/kilogram of the subject's weight.

The N6-benzylaminopurine may be present in the administrable form in a concentration range from about 150 to about 300 milligrams/kilogram of the subject's weight.

The N6-benzylaminopurine may be present in the administrable form in a concentration of about 200 milligrams/kilogram of the subject's weight.

The N6-benzylaminopurine may be in an ingestible solid tablet form.

The N6-benzylaminopurine may be in a powder form encased in an ingestible capsule.

The N6-benzylaminopurine may be in the form of a liquid. The liquid may be any in which that N6-benzylaminopurine is soluble. The liquid may include a solution of 0.02 M NaOH with 2% erythritol or water.

The liquid may be formulated as an administrable intranasal spray.

The N6-benzylaminopurine may be in the form of an ingestible emulsion. The emulsion may comprise a 50% honey format with about 0.5% carboxymethyl cellulose.

The appetite suppressant N6-benzylaminopurine may be used in combination with any other weight loss compounds.

The N6-benzylaminopurine may be used in combination with any other diabetes medications, including insulin sensitizing compounds.

The N6-benzylaminopurine may be used as a treatment for obesity-related diseases, including diabetes, heart disease, depression, fatty liver disease, diabetes-related comorbidities, and polycystic ovarian disease.

The N6-benzylaminopurine may be used in combination with any other therapeutic compounds known to increase appetite as a side effect.

The N6-benzylaminopurine may be used as an inhibitor of AMP-kinase.

The N6-benzylaminopurine may be used as an inhibitor of mTORC1.

The N6-benzylaminopurine may be used as an inhibitor of mTORC1acting through induction of Sestrin2 and Tuberous Sclerosis Complex 2 (TSC2).

The N6-benzylaminopurine may be used as an inhibitor of neuropeptide Y (NPY).

The N6-benzylaminopurine may be used as an inhibitor of neuropeptide Y acting through reduction of S6K phosphorylation and/or changes in transcription factors Early Growth Response Proteins 1 and 3 (Egr1 and Egr3).

The N6-benzylaminopurine may be used as an inducer of proopiomelanocortin (POMC).

The N6-benzylaminopurine may be used as an inducer of Akt phosphorylation and PI3 kinase.

The N6-benzylaminopurine may be used as a repressor of ERK1/2 phosphorylation, a member of the mitogen-activated protein (MAP) kinase family.

The N6-benzylaminopurine may be used as a modulator of nucleoside diphosphate kinase (NDPK).

The N6-benzylaminopurine may be used as an inducer of insulin receptor.

The N6-benzylaminopurine may be used to enhance glucose tolerance and improve insulin sensitivity.

The related cytokinin from the same family kinetin may also be used as an inhibitor of Npy expression thus may also act as an appetite suppressant.

The subject animal may be any one of humans, human pets and farm animals.

A further understanding of the functional and advantageous aspects of the disclosure can be realized by reference to the following detailed description and drawings.

### BRIEF DESCRIPTION OF THE DRAWINGS

Embodiments for the use of N6-Benzylaminopurine (BAP) as an appetite suppressant in animals will now be described, by way of example only, with reference to the drawings, in which:

FIG. 1A is a plot of the body weight of the mice in grams on the Y axis and the days of exposure to the treatment on the X axis. Body weight of male mice exposed to BAP is reduced. Continuous exposure (33 days) of 10 mM BAP solution in male CD-1 mice on a 60% HFD on body weight. Two-way ANOVA, n=8 per group, *p<0.05, **p<0.01, ***p<0.001, ****p<0.0001.

FIG. 1B is a plot of the body weight as a percentage of starting weight of the mice on the Y axis and the days of exposure to the treatment on the X axis. Body weight of male mice exposed to BAP is reduced. Continuous exposure (33 days) of 10 mM BAP solution in male CD-1 mice on a 60% HFD on body weight where body weight is represented as a percentage of starting weight. Two-way ANOVA, n=8 per group, *p<0.05, **p<0.01, ***p<0.001, ****p<0.0001.

FIG. 1C is a plot of the body weight of the mice in grams on the Y axis and the days of exposure to the treatment on the X axis. Continuous exposure (33 days) of 10 mM BAP solution in male CD-1 mice on a sucrose-matched control diet on body weight, Two-way ANOVA, n=8 per group, *p<0.05, **p<0.01, ***p<0.001, ****p<0.0001.

FIG. 1D is a plot of the body weight as a percentage of starting weight of the mice on the Y axis and the days of exposure to the treatment on the X axis. Body weight of male mice exposed to BAP is reduced. Continuous exposure (33 days) of 10 mM BAP solution in male CD-1 mice on a sucrose-matched control diet on body weight where body weight is represented as a percentage of starting weight. Two-way ANOVA, n=8 per group, *p<0.05, **p<0.01, ***p<0.001, ****p<0.0001.

FIG. 2A is a plot of the water intake of the mice in milliliters on the Y axis and the days of exposure to the treatment on the X axis. Water intake of male CD-1 mice exposed to BAP in the water supplemented with erythritol sweetener is significantly less than control. Continuous exposure (33 days) of 10 mM BAP solution in male CD-1 mice on a 60% HFD in water intake. Two-way ANOVA, n=3 per group, *p<0.05, ****p<0.0001. Total water intake, unpaired t-test, n=3, *p<0.05.

FIG. 2B is a plot of the water intake of the mice in milliliters on the Y axis and the days of exposure to the treatment on the X axis. Water intake of male CD-1 mice exposed to BAP in the water supplemented with erythritol sweetener is significantly less than control. Continuous exposure (33 days) of 10 mM BAP solution in male CD-1 mice on a sucrose-matched control diet. Two-way ANOVA, n=3 per group, *p<0.05, ****p<0.0001. Total water intake, unpaired t-test, n=3, *p<0.05.

FIG. 3 is a plot of blood glucose level (mmol/L) versus time in minutes for mice exposed to BAP after an intraperitoneal insulin tolerance test. Male CD-1 display improved glucose tolerance after 28 days of daily oral BAP exposure on a 60% HFD. Unpaired Student's t-test, n=8, *p<0.05, **p<0.01.

FIG. 4A are plots of the levels of gene expression on the Y axis versus the treatment on the X axis. Male CD-1 mice exposed to BAP did not exhibit altered hypothalamic feeding neuropeptide expression on the 60% HFD. Hypothalamic feeding neuropeptide expression after 33 days of exposure to BAP or vehicle control on a HFD Results expressed as changes using an unpaired Student's t-test, n=8-11. Npy—neuropeptide Y, Agrp—agouti-related peptide, Pomc—proopiomelanocortin are all expressed relative to the Rpl7 control gene.

FIG. 4B are plots of the levels of gene expression on the Y axis versus the treatment on the X axis. Male CD-1 mice exposed to BAP exhibited altered hypothalamic feeding neuropeptide expression on the sucrose-matched control diet. Hypothalamic feeding neuropeptide expression after 33 days of exposure to BAP or vehicle control on a sucrose-matched control diet. Results expressed as changes using an unpaired Student's t-test, n=8-11, **p<0.01. Npy—neuropeptide Y, Agrp—agouti-related peptide, Pomc—proopiomelanocortin are all expressed relative to the Rpl7 control gene.

FIG. 5A is a plot of the body weight of the mice in grams on the Y axis and the days of exposure to the treatment on the X axis. Body weight of female mice exposed to BAP is reduced. Continuous exposure (33 days) of 10 mM BAP solution or vehicle control in female CD-1 mice on a 60% HFD on body weight. Two-way ANOVA, n=8 per group.

FIG. 5B is a plot of the body weight as a percentage of starting weight of the mice on the Y axis and the days of exposure to the treatment on the X axis. Body weight of female mice exposed to BAP is reduced. Continuous exposure (33 days) of 10 mM BAP solution or vehicle control in female CD-1 mice on a 60% HFD on body weight where body weight is represented as a percentage of starting weight. Two-way ANOVA, n=7-10 per group, *p<0.05.

FIG. 5C is a plot of the body weight of the mice in grams on the Y axis and the days of exposure to the treatment on the X axis. Continuous exposure (33 days) of 10 mM BAP solution or vehicle control in female CD-1 mice on a sucrose-matched control diet on body weight, Two-way ANOVA, n=7-10 per group.

FIG. 5D is a plot of the body weight as a percentage of starting weight of the mice on the Y axis and the days of exposure to the treatment on the X axis. Body weight of female mice exposed to BAP is not reduced on the control diet. Continuous exposure (33 days) of 10 mM BAP solution or vehicle control in female CD-1 mice on a sucrose-matched control diet on body weight where body weight is represented as a percentage of starting weight. Two-way ANOVA, n=7-10 per group.

FIG. 6A is a plot of the water intake of the mice in milliliters on the Y axis and the days of exposure to the treatment on the X axis. Water intake of female CD-1 mice exposed to BAP in the water supplemented with erythritol sweetener is decreased compared to control across diets. Continuous exposure (33 days) of 10 mM BAP solution or vehicle control in female CD-1 mice on a 60% HFD had decreased water intake. Unpaired t-test, n=3 per group for HFD, ****p<0.0001.

FIG. 6B is a plot of the water intake of the mice in milliliters on the Y axis and the days of exposure to the treatment on the X axis. Water intake of female CD-1 mice exposed to BAP in the water supplemented with erythritol sweetener is decreased compared to control in sucrose-matched control diet. Continuous exposure (33 days) of 10 mM BAP solution or vehicle control in female CD-1 mice on a sucrose-matched control diet. n=2-3 for sucrose-matched control (no statistics).

FIG. 7 is a plot of blood glucose level (mmol/L) versus time in minutes in female mice exposed to BAP that demonstrates they do not exhibit enhanced insulin sensitivity. Intraperitoneal insulin tolerance test of female CD-1 mice exposed to a HFD with BAP compared to control after 28 days of exposure. Unpaired Student's t-test, n=8-10.

FIG. 8A are plots of the levels of gene expression on the Y axis versus the treatment on the X axis. Female CD-1 mice exposed to BAP did not exhibit altered hypothalamic feeding neuropeptide expression of Npy and Agrp on the 60% HFD, but had a significant decrease in Pomc expression. Hypothalamic feeding neuropeptide expression after 33 days of exposure to BAP or vehicle control on a HFD Results expressed as changes using an unpaired Student's t-test, n=7-10, *p<0.05. Npy—neuropeptide Y, Agrp—agouti-related peptide, Pomc—proopiomelanocortin are all expressed relative to the Rpl7 control gene.

FIG. 8B are plots of of the levels of gene expression on the Y axis versus the treatment on the X axis. Female CD-1 mice exposed to BAP did not exhibit altered hypothalamic feeding neuropeptide expression on the sucrose-matched control diet. Hypothalamic feeding neuropeptide expression after 33 days of exposure to BAP or vehicle control on a sucrose-matched control diet. Results expressed as changes using an unpaired Student's t-test, n=7-10. Npy—neuropeptide Y, Agrp—agouti-related peptide, Pomc—proopiomelanocortin are all expressed relative to the Rpl7 control gene.

FIG. 9A (cohort CCBR) is a plot of the body weight of the mice in grams on the Y axis and the days of exposure to the treatment on the X axis. Body weight of male mice exposed to BAP is reduced. Daily exposure (11 days) of 300 mg/kg BAP emulsion or the control emulsion (vehicle) in male CD-1 mice on a 60% HFD on body weight.; and sucrose-matched control diet

Two-way ANOVA, n=10 per group, *p<0.05, **p<0.01, ***p<0.001, ****p<0.0001.

FIG. 9B (cohort CCBR) is a plot of the body weight as a percentage of starting weight of the mice on the Y axis and the days of exposure to the treatment on the X axis. Body weight of male mice exposed to BAP is reduced. Daily exposure (11 days) of 300 mg/kg BAP emulsion or the control emulsion (vehicle) in male CD-1 mice on a 60% HFD on body weight as represented as a percentage of starting weight. Two-way ANOVA, n=10 per group, *p<0.05, **p<0.01, ***p<0.001, ****p<0.0001.

FIG. 10A (cohort CCBR) is a plot of peripheral fat area on the Y axis and treatment regimen on the X axis No change is detected in fat mass over the short-term. Male CD-1 mice on a HFD exposed to BAP emulsion or the control emulsion (vehicle) for 11 days do not have any detectable changes in net peripheral fat mass by Bruker scan, n=10. Mice were measured by Bruker scan before and after 11 days of treatment.

FIG. 10B (cohort CCBR) is a plot of peripheral fat area percentage on the Y axis and treatment regimen on the X axis No change is detected in fat mass over the short-term. Male CD-1 mice on a HFD exposed to BAP emulsion or the control emulsion (vehicle) for 11 days do not have any detectable changes in the percentage of peripheral fat mass by Bruker scan, n=10. Mice were measured by Bruker scan before and after 11 days of treatment.

FIG. 11 (cohort CCBR) is a plot of food intake in grams versus days of exposure to BAP demonstrating that food intake is altered with exposure to BAP emulsion or the control emulsion (vehicle). Male CD-1 mice on a 60% HFD exposed to BAP emulsion for 11 days significantly decreased food intake compared to control. Two-way ANOVA, n=5, *p<0.05, **p<0.01.

FIG. 12 (cohort CCBR) is a plot of water intake in milliliters that reveals that no change is detected in water consumption with BAP emulsion versus the control emulsion (vehicle). Male CD-1 mice on a 60% HFD exposed to BAP emulsion or the control emulsion (vehicle) for 11 days do not have any detectable changes in water intake, n=5.

FIG. 13 (cohort CCBR) shows that male CD-1 mice exposed to BAP emulsion exhibited altered neuropeptide gene expression on the y-axis. Hypothalamic feeding neuropeptide expression in male CD-1 mice fed a HFD after 11 days of exposure to the BAP emulsion. Npy and Agrp mRNA is altered by BAP exposure compared to vehicle control. Results expressed as changes using an unpaired Student's t-test, n=10, **p<0.01 (n=10). Npy—neuropeptide Y, Agrp—agouti-related peptide, Pomc—proopiomelanocortin are all expressed relative to the Rpl7 control gene.

FIG. 14A (cohort BSF) is a plot of the body weight of the mice in grams on the Y axis and the days of exposure to the treatment on the X axis. Body weight of male mice exposed to BAP is reduced. Daily exposure (14 days) of 300 mg/kg BAP emulsion or the control emulsion (vehicle) in male CD-1 mice on a 60% HFD on body weight. Two-way ANOVA, n=22 per group, *p<0.05, **p<0.01, ***p<0.001, ****p<0.0001.

FIG. 14B (cohort BSF) is a plot of the body weight as a percentage of starting weight of the mice on the Y axis and the days of exposure to the treatment on the X axis. Body weight of male mice exposed to BAP is reduced. Daily exposure (14 days) of 300 mg/kg BAP emulsion or the control emulsion (vehicle) in male CD-1 mice on a 60% HFD on body weight as represented as a percentage of starting weight. Two-way ANOVA, n=22 per group, *p<0.05.

FIG. 15 (cohort BSF) is a plot of food intake in grams versus days of exposure to BAP demonstrating that food intake is not altered with exposure to BAP emulsion or the control emulsion (vehicle). Male CD-1 mice on a 60% HFD exposed to BAP emulsion for 14 days have no change in food intake. Two-way ANOVA, n=9-10.

FIG. 16 (cohort BSF) is a plot of water intake in milliliters that reveals that no change is detected in water consumption with BAP emulsion versus the control emulsion (vehicle). Male CD-1 mice on a 60% HFD exposed to BAP emulsion, or the control emulsion (vehicle) for 14 days do not have any detectable changes in water intake. Two-way ANOVA, n=9-10.

FIG. 17 (cohort BSF) shows that male CD-1 mice exposed to BAP emulsion exhibited altered neuropeptide gene expression on the y-axis. Hypothalamic feeding neuropeptide expression in male CD-1 mice fed a HFD after 14 days of exposure to the BAP emulsion. Npy and Agrp mRNAs are altered by BAP exposure compared to vehicle control. Results expressed as changes using an unpaired Student's t-test, n=14, *p<0.05. Npy—neuropeptide Y, Agrp—agouti-related peptide, Pomc—proopiomelanocortin are all expressed relative to the Rpl7 control gene.

FIG. 18 (cohort BSF) is a plot of blood glucose level (mmol/L) versus time in minutes for mice exposed to BAP after an intraperitoneal glucose injection. Male CD-1 mice did not have improved glucose tolerance after 14 days of daily oral BAP exposure. Two-way ANOVA, n=8.

FIG. 19A (cohort BSF) is a plot of the body weight of the mice in grams on the Y axis and the days of exposure to the treatment on the X axis. Body weight of male mice exposed to BAP is reduced. Daily exposure (25days) of 300 mg/kg BAP emulsion or the control emulsion (vehicle) in male CD-1 mice on a 60% HFD on body weight. Two-way ANOVA, n=16 per group, *p<0.05.

FIG. 19B (cohort BSF) is a plot of the body weight as a percentage of starting weight of the mice on the Y axis and the days of exposure to the treatment on the X axis. Body weight of male mice exposed to BAP is reduced. Daily exposure (25 days) of 300 mg/kg BAP emulsion or the control emulsion (vehicle) in male CD-1 mice on a 60% HFD on body weight as represented as a percentage of starting weight. Two-way ANOVA, n=16 per group, *p<0.05, **p<0.01, ***p<0.001.

FIG. 20A (cohort BSF) is a plot of food intake in grams versus days of exposure to BAP demonstrating that food intake is decreased with exposure to BAP emulsion over time versus the control emulsion (vehicle). Two-way ANOVA, n=6-8, *p<0.05, **p<0.01, ***p<0.001.

FIG. 20B (cohort BSF) is a plot of average daily food intake on the Y axis versus treatment on the X axis. Male CD-1 mice on a 60% HFD exposed to BAP emulsion for 24 days have significant changes in food intake. 2-way ANOVA, n=6-8, ***p<0.001.

FIG. 21 (cohort BSF) is a plot of water intake in milliliters reveals that no change is detected in water consumption with BAP emulsion versus the control emulsion (vehicle). Male CD-1 mice on a 60% HFD exposed to BAP emulsion, or the control emulsion (vehicle) for 24 days do not have any detectable changes in water intake, n=6-8.

FIG. 22 (cohort BSF) shows that male CD-1 mice exposed to BAP emulsion exhibited altered neuropeptide gene expression on the y-axis. Hypothalamic feeding neuropeptide expression in male CD-1 mice fed a HFD after 27 days of exposure to the BAP emulsion. Npy and Agrp mRNAs are altered by BAP exposure compared to vehicle control. Results expressed as changes using an unpaired Student's t-test, n=6-8, **p<0.01. Npy—neuropeptide Y, Agrp—agouti-related peptide, Pomc—proopiomelanocortin are all expressed relative to the Rpl7 control gene.

FIG. 23A (cohort BSF) is a plot of blood glucose level (mmol/L) versus time in minutes after intraperitoneal glucose injection for a male CD-1 mice exposed to BAP. Male CD-1 mice exposed to BAP have improved glucose tolerance after 27 days of daily oral BAP exposure after an intraperitoneal glucose tolerance test. Two-way ANOVA, n=8, *p<0.05, ***p<0.001.

FIG. 23B (cohort BSF) is a plot of blood glucose level (mmol/L) versus time in minutes for mice exposed to BAP with the baseline subtracted after an intraperitoneal glucose tolerance test. Two-way ANOVA, n=8, *p<0.05.

FIG. 23C (cohort BSF) is a plot of blood glucose level (mmol/L) in male CD-1 mouse exposed to BAP as represented by the area of the curve quantification from the plot in FIG. 23B. Unpaired Student's t-test, n=8, *p<0.05, **p<0.01.

FIG. 24A (cohort BSF) is a plot of the body weight of the mice in grams on the Y axis and the days of exposure to the treatment on the X axis. Body weight of male mice exposed to BAP is reduced. Daily exposure (14 days) of 75, 150, 300 mg/kg BAP emulsion or the control emulsion (vehicle) in male CD-1 mice on a 60% HFD on body weight. Two-way ANOVA, n=13-14 per group.

FIG. 24B (cohort BSF) is a plot of the body weight as a percentage of starting weight of the mice on the Y axis and the days of exposure to the treatment on the X axis. Body weight of male mice exposed to BAP is reduced. Continuous exposure (14 days) of 75, 150, 300 mg/kg BAP emulsion or the control emulsion (vehicle) in male CD-1 mice on a 60% HFD on body weight as represented as a percentage of starting weight. Only the 150, 300 mg/kg BAP were significantly different from control. Two-way ANOVA, n=13-14 per group, *p<0.05, **p<0.01, ***p<0.001.

FIG. 25A (cohort BSF) is a plot of food intake in grams versus days of exposure to BAP demonstrating that food intake is not altered with exposure to BAP emulsion or the control emulsion (vehicle). Two-way ANOVA, n=5-7

FIG. 25B (cohort BSF) is a plot of cumulative daily food intake on the Y axis versus treatment on the X axis. Male CD-1 mice on a 60% HFD exposed to BAP emulsion for 14 days do not have significant changes in food intake, although the 300 mg/kg BAP approached significance. Two-way ANOVA, n=5-7.

FIG. 26 (cohort BSF) is a plot of water intake in milliliters that reveals that no change is detected in water consumption with BAP emulsion versus the control emulsion (vehicle). Male CD-1 mice on a 60% HFD exposed to BAP emulsion, or the control emulsion (vehicle) for 12 days do not have any detectable changes in water intake. Two-way ANOVA, n=5-7.

FIG. 27A (cohort CCBR) is a plot of body weight in grams versus dates for female mice showing female mice did not continue to gain weight on a 60% HFD due to stress because of noise at the animal facility.

FIG. 27B (cohort CCBR) is a plot of food intake (grams) versus days of exposure for female mice showing female mice did not eat more on a 60% HFD due to stress because of noise at the animal facility. The female mice were fed BAP emulsion or the control emulsion (vehicle) for 12 days but ate less honey emulsion even before the addition of BAP. The experiment was therefore terminated.

FIG. 28A (cohort BSF) is a plot of the body weight of the mice in grams on the Y axis and the days of exposure to the treatment on the X axis. Body weight of female mice exposed to BAP does not increase. Daily exposure (36 days) of 300 mg/kg BAP emulsion or the control emulsion (vehicle) in female CD-1 mice on a 60% HFD on body weight. Two-way ANOVA, n=9 per group.

FIG. 28B (cohort BSF) is a plot of the body weight as a percentage of starting weight of the mice on the Y axis and the days of exposure to the treatment on the X axis. Body weight of female mice exposed to BAP does not increase with 60% HFD. Daily exposure (36 days) of 300 mg/kg BAP emulsion or the control emulsion (vehicle) in female CD-1 mice on a 60% HFD on body weight. Two-way ANOVA, n=9 per group.

FIG. 29 (cohort BSF) is a plot of food intake in grams versus days of exposure to BAP demonstrating that food intake is not altered with exposure to BAP emulsion or the control emulsion (vehicle). Female CD-1 mice on a 60% HFD exposed to BAP emulsion for 36 days have no change in food intake. Two-way ANOVA, n=2-3.

FIG. 30 (cohort BSF) is a plot of water intake in milliliters that reveals that no change is detected in water consumption with BAP emulsion versus the control emulsion (vehicle). Female CD-1 mice on a 60% HFD exposed to BAP emulsion for 36 days have no change in water intake. Two-way ANOVA, n=2-3.

FIG. 31 are plots of the levels of gene expression on the Y axis versus the treatment on the X axis. Female CD-1 mice exposed to 300 mg/kg BAP emulsion did not exhibit altered hypothalamic feeding neuropeptide expression on the 60% HFD versus control. Hypothalamic feeding neuropeptide expression after 36 days of exposure to BAP or vehicle control on a HFD. Results expressed as changes using an unpaired Student's t-test, n=7. Npy—neuropeptide Y, Agrp—agouti-related peptide, Pomc—proopiomelanocortin are all expressed relative to the Rpl7 control gene.

FIG. 32A are plots of the levels of gene expression on the Y axis versus the treatment on the X axis. Only Pomc gene expression is induced in CD-1 mouse-derived hypothalamic primary culture. A 16 hr BAP treatment (100 μM) versus the vehicle control in male-derived primary cultured hypothalamic neurons assessed changes in hypothalamic feeding-related neuropeptide expression using an unpaired Student's t-test, n=7, ***p<0.001, ****p<0.0001. Npy—neuropeptide Y, Agrp—agouti-related peptide, Pomc—proopiomelanocortin are all expressed relative to the Rpl7 control gene.

FIG. 32B are plots of the levels of gene expression on the Y axis versus the treatment on the X axis. Only Pomc gene expression is induced in CD-1 mouse-derived hypothalamic primary culture. A 16 hr BAP treatment (100 μM) versus the vehicle control in female-derived primary cultured hypothalamic neurons assessed changes in hypothalamic feeding-related neuropeptide expression using an unpaired Student's t-test, n=7, ***p<0.001, ****p<0.0001. Npy—neuropeptide Y, Agrp—agouti-related peptide, Pomc—proopiomelanocortin are all expressed relative to the Rpl7 control gene.

FIG. 33A is a plot of the levels of gene expression on the Y axis versus the cell line assesed on the X axis. Multiple hypothalamic neuronal cell lines treated with 100 μM BAP or vehicle control for 16 hr. Hypothalamic feeding neuropeptide Npy expression is assessed in mHypoE-46 (n=4), mHypoE-44 (n=4), mHypoA-Bmal1-WT/F (n=3), mHypoE-38 (n=3), mHypoA-59 (n=4), and mHypoE-41 (n=4) cell lines. Control treatment is represented by the dotted line set to one. Unpaired Student's t-test, *p<0.05, **p<0.01, ***p<0.001, *** *p<0.0001. Npy—neuropeptide Y is expressed relative to the Rpl7 control gene.

FIG. 33B is a plot of the levels of gene expression on the Y axis versus the cell line assesed on the X axis. Multiple hypothalamic neuronal cell lines treated with 100 μM BAP or vehicle control for 16 hr. Hypothalamic feeding neuropeptide Agrp expression is assessed in mHypoE-46 (n=4), mHypoE-44 (n=4), mHypoA-Bmal1-WT/F (n=3), mHypoE-38 (n=3), mHypoA-59 (n=4), and mHypoE-41 (n=4) cell lines. Control treatment is represented by the dotted line set to one. Unpaired Student's t-test, *p<0.05, **p<0.01, ***p<0.001, ****p<0.0001. Agrp—agouti-related peptide is expressed relative to the Rpl7 control gene.

FIG. 33C is a plot of the levels of gene expression on the Y axis versus the cell line assesed on the X axis. Hypothalamic neuronal cell lines treated with 100 μM BAP or vehicle control for 16 hr. Hypothalamic feeding neuropeptide Pomc expression is assessed in mHypoA-Bmal1-WT/F (n=3) and mHypoA-POMC/GFP-2 (n=3) cell lines. For the mHypoA-Bmal1-WT/F result, unpaired Student's t-test, *p<0.05, **p<0.01. For the mHypoA-POMC/GFP-2 results, two-way ANOVA, *p<0.05, **p<0.01, ***p<0.001. Pomc—proopiomelanocortin is expressed relative to the Rpl7 control gene.

FIG. 34A is a plot of the levels of gene expression on the Y axis versus the time of treatment on the X axis. Timecourse over 24 hr with 100 μM BAP in the mHypoE-46 hypothalamic cell line. Time-matched control treatments are represented by white bars set to one. Npy expression in mHypoE-46, n=4. Two-way ANOVA, *p<0.05, **p<0.01. Npy—neuropeptide Y is expressed relative to the Rpl7 control gene.

FIG. 34B is a plot of the levels of gene expression on the Y axis versus the time of treatment on the X axis. Timecourse over 24 hr with 100 μM BAP in the mHypoE-44 hypothalamic cell line. Time-matched control treatments are represented by the white bar set to one. Npy expression in mHypoE-44, n=3. Two-way ANOVA, *p<0.05, **p<0.01, ***p<0.001. Npy—neuropeptide Y is expressed relative to the Rpl7 control gene.

FIG. 35 are plots of the levels of gene expression on the Y axis versus the treatment dose on the X axis. Dose curve with increasing concentrations of BAP or vehicle control treatment for 16 hr in hypothalamic cell lines. Npy expression is assessed in mHypoE-46 (n=3), mHypoE-44 (n=4), and mHypoE-41 (n=4) cell lines. One-way ANOVA, *p<0.05, **p<0.01, ***p<0.001, ****p<0.0001. Npy—neuropeptide Y is expressed relative to the Rpl7 control gene.

FIG. 36 are plots of the levels of gene expression on the Y axis versus the treatment condition on the X axis. Co-treatment with or without 100 μM ATP and 100 μM BAP or vehicle control for 16 hr in Npy-expressing cell lines. Npy mRNA expression is assessed in mHypoE-46, mHypoE-44, and mHypoE-41 cell lines. Two-way ANOVA, n=4, *p<0.05, **p<0.01, ***p<0.001, ***p<0.001. Npy—neuropeptide Y is expressed relative to the His control gene.

FIG. 37 are plots of the levels of gene expression on the Y axis versus the treatment condition on the X axis. Co-treatment with or without 100 μM UDP and 100 μM BAP or vehicle control for 16 hr in Npy-expressing cell lines. Npy mRNA expression is assessed in mHypoE-46 and mHypoE-44 cell lines. Two-way ANOVA, n=4, *p<0.05, **p<0.01, ***p<0.001, ***p<0.001. Npy—neuropeptide Y is expressed relative to the Rpl7 control gene.

FIG. 38 are plots of the levels of gene expression on the Y axis versus the treatment condition on the X axis. Co-treatment with or without 100 μM UTP and 100 μM BAP or vehicle control for 16 hr in Npy-expressing cell lines. Npy mRNA expression is assessed in mHypoE-46 and mHypoE-44 cell lines. Only n=2 due to lack of effect with the UTP (no statistics), preliminary data. Npy—neuropeptide Y is expressed relative to the Rpl7 control gene.

FIG. 39 are plots of the levels of gene expression on the Y axis versus the treatment condition on the X axis. Co-treatment with or without 10 μM PKI or 1 μM H89 (as indicated) and 100 μM BAP or vehicle control for 16 hr in Npy-expressing cell lines. Npy mRNA expression is assessed in mHypoE-46 and mHypoE-44 cell lines (as indicated). Only n=2 due to lack of effect with the inhibitors (no statistics). Npy—neuropeptide Y is expressed relative to the Rpl7 control gene.

FIG. 40 is a plot of the levels of gene expression on the Y axis versus the treatment condition on the X axis. Co-treatment with or without 50 μM palmitate and 100 μM BAP or vehicle control for 16 hr in the mHypoE-46 cell line. Two-way ANOVA, n=4, ****p<0.0001. Npy—neuropeptide Y is expressed relative to the Rpl7 control gene.

FIG. 41A is a plot of the phosphorylation levels of AMP-kinase (AMPK) on the Y axis and the treatment on the X axis. Treatment with 100 μM BAP for 15 min in the mHypoE-46 cell line results in a decrease in phosphorylation of AMPK versus treatment with vehicle alone. A representative Western blot image with two of the three experiments imaged is included below the graph with phospho-AMPK and total AMPK bands indicated. Student's t-test, n=3, **p<0.01.

FIG. 41B is a plot of the phosphorylation levels of AMP-kinase (AMPK) on the Y axis and the treatment on the X axis. Treatment with either

AMPK activator AICAR and no glucose medium (also an activator control) causes an increase in AMPK phosphorylation after 15 min in the mHypoE-46 cell line versus treatment with vehicle alone. A representative Western blot image with two of the three experiments imaged is included below the graph with phospho-AMPK and total AMPK bands indicated. Student's t-test, n=3, *p<0.05, **p<0.01.

FIG. 42A is a plot of the levels of gene expression on the Y axis versus the cell line assesed on the X axis. Hypothalamic neuronal cell line mHypoE-46 treated with 100 UM BAP or vehicle control for 4 hr to validate a number of genes that changed in the RNAseq analysis. Vehicle control treatment is represented by the dotted line set to one. Two-way ANOVA, n=4-6, *p<0.05, **p<0.01, ***p<0.001, ****p<0.0001. All genes listed are expressed relative to the Rpl7 control gene.

FIG. 42B is a plot of the levels of gene expression on the Y axis versus the cell line assesed on the X axis. Hypothalamic neuronal cell line mHypoE-46 treated with 100 μM BAP or vehicle control for 16 hr to validate a number of genes that changed in the RNAseq analysis. Vehicle control treatment is represented by the dotted line set to one. Two-way ANOVA, n=4-6, *p<0.05, **p<0.01, ***p<0.001, ****p<0.0001. All genes listed are expressed relative to the Rpl7 control gene.

FIG. 43 is a plot of the phosphorylation levels of phospho-p70-S6K (Thr389) on the Y axis and the treatment on the X axis. The neurons were exposed to 100 μM BAP for 5, 15, 30, 60 and 90 minutes followed by protein isolation and Western blot analysis to assess phosphorylation of p70-S6K versus vehicle alone. A representative Western blot image with one of the four experiments imaged is included below the graph with phospho-p70-S6K and total 70-S6K bands indicated. Two-way ANOVA, n=4, *p<0.05, **p<0.01.

FIG. 44 is a plot of the phosphorylation levels of phosphorylation of ERK1/2 (Thr202/Tyr204) on the Y axis and the treatment on the X axis. The neurons were exposed to 100 μM BAP for 5 minutes followed by protein isolation and Western blot analysis to assess phosphorylation of ERK1/2 versus vehicle alone. A representative Western blot image with one of the four experiments imaged is included below the graph with phospho-ERK1/2 and total ERK1/2 bands indicated. Unpaired Student's t-test, n=5, *p<0.05.

FIG. 45 is a plot of the levels of gene expression on the Y axis versus the cell line assesed on the X axis. Multiple hypothalamic neuronal cell lines treated with 100 μM BAP or vehicle control for 16 hr. Ndpk1and Ndpk2 are assessed in the mHypoE-46 (n=4), mHypoE-44 (n=3), and mHypoE-41 (n=3) cell lines. Control treatment is represented by the dotted line set to one. Unpaired Student's t-test, *p<0.05, **p<0.01, ***p<0.001, ****p<0.0001. Ndpk1-nucleoside diphosphate kinase 1 and Ndpk2-nucleoside diphosphate kinase 2 is expressed relative to the Rpl7 control gene.

FIG. 46 is a plot of the phosphorylation levels of Akt (protein kinase B) on the Y axis and the treatment on the X axis. The Akt phosphorylation level is represented as the fold change of phospho-AKT to total AKT ratio in the insulin-rechallenged samples relative to PBS-rechallenged controls (set to one). Treatment with 100 UM BAP for 24 h in the mHypoE-46 cell line results in an increase in phosphorylation of Akt versus treatment with the DMSO vehicle alone. Cellular insulin resistance was induced by insulin pre-treatment (100 μM, 24 h), represented by the hatched bars. There is no change in Akt phosphorylation in response to cellular insulin resistance with BAP compared to vehicle control. A representative Western blot image with one of the four experiments imaged is included below the graph with phospho-Akt and total Akt bands indicated. Two-way ANOVA, n=4, *p<0.05, **p<0.01, ***p<0.001, ****p<0.0001.

FIG. 47A is a plot of the levels of gene expression on the Y axis versus the cell line assesed on the X axis. mHypoE-46 neuronal cell line treated with 100 μM BAP or vehicle control for 16 hr. Insulin receptor β subunit (Insrb) gene expression is assessed by qRT-PCR. Unpaired Student's t-test, n=3, **p<0.01. Insrb-InsR beta subunit is expressed relative to the Rpl7 control gene.

FIG. 47B is a plot of the protein levels of insulin receptor ß subunit (InsR) on the Y axis and the treatment on the X axis. Treatment with 100 mM BAP for 24 h in the mHypoE-46 cell line increases the InsR protein level versus treatment with the vehicle alone. Cellular insulin resistance was induced by insulin pre-treatment (100 μM, 24 h), represented by the hatched bars. There is no change in InsR protein level in response to cellular insulin resistance with BAP relative to vehicle control. A representative Western blot image with one of the four experiments imaged is included below the graph with InsR and the loading control alpha tubulin (a-Tu) bands indicated. One way ANOVA, n=4, *p<0.05, **p<0.01, ***p<0.001, ****p<0.0001.

FIG. 48 is a plot of the levels of gene expression on the Y axis versus the cell line assesed on the X axis. mHypoE-46 neuronal cell line treated with 100 μM kinetin or vehicle control for 16 hr. Neuropeptide Y (Npy) gene expression is assessed by qRT-PCR. Preliminary data, n=2 (no statistics). Npy—neuropeptide Y is expressed relative to the Rpl7 control gene.

### DETAILED DESCRIPTION

Various embodiments and aspects of the disclosure involving the use of N6-benzylaminopurine (BAP) as an appetite suppressant in animals will be described with reference to details discussed below. The following description and drawings are illustrative of the disclosure and are not to be construed as limiting the disclosure. The drawings are not necessarily to scale. Numerous specific details are described to provide a thorough understanding of various embodiments of the present disclosure. However, in certain instances, well-known or conventional details are not described in order to provide a concise discussion of embodiments of the present disclosure.

As used herein, the terms, “comprises” and “comprising” are to be construed as being inclusive and open ended, and not exclusive. Specifically, when used in this specification including claims, the terms, “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately”, when used in conjunction with ranges of dimensions of particles, compositions of mixtures or other physical properties or characteristics, are meant to cover slight variations that may exist in the upper and lower limits of the ranges of dimensions so as to not exclude embodiments where on average most of the dimensions are satisfied but where statistically dimensions may exist outside this region. It is not the intention to exclude embodiments such as these from the present disclosure.

As used herein, “subject” refers to animals including but not limited to mammals such as, but not limited to, domestic pets (dogs, cats etc.), farm animals and humans to mention some non-limiting examples.

Exemplary studies of the use of N6-benzylaminopurine (BAP) as an appetite suppressant in mice are presented hereinafter.

### Methods

BAP in the drinking water in CD-1 mice.

All protocols using mice were approved by the University Animal Care Committee of the University of Toronto and were performed in accordance with the Canadian Council on Animal Care. Animal experiments were performed in three separate animal facilities: Medical Sciences Building (MSB), Centre for Cellular and Biomolecular Research (CCBR) and the Biological Sciences Facility (BSF) due to ongoing construction and housing changes. Facilities and dates are indicated in each experimental cohort. N6-benzylaminopurine (BAP; also called N6-benzyladenine) was obtained from Sigma (B3408, cell culture grade in powder form; CAS number 1214-39-7). Male and female CD-1 mice aged 6-7 weeks were acquired from Charles Rivers and acclimated on a chow diet for one week prior to experimentation. Mice were then weight-matched and placed on either a 60% high fat diet (HFD) or sucrose-matched control diet for 4 weeks (from Research Diets: 60% HFD D12492 and the sucrose-matched control diet D12450J). After 3 weeks on the diet, mice were acclimated for one week to the drinking apparatus, a 50 mL canonical tube with a drinking tube. After 4 weeks on the diet, the mice were again weight-matched and randomly assigned to either the control or BAP group. The control group and BAP group had their drinking water replaced with either a solution of 0.02 M NaOH with 2% erythritol or 10 mM BAP in 0.02 M NaOH with 2% erythritol, respectively. The average dosages achieved using this method are as follows: male HFD (200 mg/kg/day), male control (157 mg/kg/day), female HFD (189 mg/kg/day), and female control (129.9 mg/kg/day). The mice were exposed continuously to the solutions for a total of 33 days. One week prior to the end of the experiment (day 28), the mice were fasted for 4 hr and underwent intraperitoneal insulin tolerance testing (ITT). After 33 days, the mice were sacrificed, and their tissues were collected. RNA was isolated from the collected hypothalamic and used to quantify gene expression via qRT-PCR.

### BAP in a Honey Emulsion Fed to CD-1 Mice.

Male and female CD-1 mice were received at age 6-7 weeks and were acclimated on a chow diet for one week prior to experimentation. After acclimation to the facility, mice were switched to a 60% HFD. The male and female mice received HFD for 4 (male) and 5 or 16 (female) weeks prior to BAP exposure. The mice were then acclimated to the emulsion feeding protocol for 4 days prior to the start of BAP exposure using the method described in Kuster et al. (12). Two days prior to the start of the exposure period, a cohort of male mice underwent acute general anesthetic with isoflurane for baseline Bruker scanning to quantify peripheral fat mass. The mice were then weight-matched and randomly assigned to either the control or BAP group. During the exposure period, mice were restrained via scruffing (held at back of neck) and fed 200 μL of an emulsion containing 0.5% carboxymethyl cellulose and 50% honey with or without BAP at 2 hr prior to lights off at the facility daily.

The BAP concentrations were calibrated weekly to achieve a dosage of 300 mg/kg/day. All mice were kept on the 60% HFD throughout the exposure period. The male mice were fed their respective emulsion for 11, 14 or 27 days (as indicated), and the female mice were fed for 12 or 36 days (as indicated). On day 12, the one cohort of male mice underwent general anesthetic for the purpose of post-experiment Bruker scanning. The male mice were then fed their assigned emulsion 2 hr prior to sacrificing and tissue collection. The female mice did not undergo a second round of Bruker scanning and were fed their assigned emulsion 16 hr prior to sacrificing. Hypothalamic collected from the mice were then used to isolate RNA and quantify gene expression using quantitative reverse transcription polymerase chain reaction (qRT-PCR).

ITT and GTT protocols.

Male mice were placed on a 60% HFD for 8 weeks prior to the ITT and GTT analyses. At 4 weeks, treatment with BAP or vehicle was introduced on a daily basis in the honey emulsion as described above. The mice were then briefly fasted to determine the response to insulin or glucose.

For the intraperitoneal insulin tolerance test (ITT), mice were transferred to clean cages without food and fasted for 4 hours. The mice were weighed after 3 hours of fasting and these weights were used to determine optimal insulin dosage (1 IU/kg). The appropriate amount of insulin was aliquoted. After 4 hours of fasting, blood glucose was measured via tail vein using a standard glucometer. This value was recorded as the 0-minute timepoint. The mice were intraperitoneal injected with their aliquoted amount of insulin and returned to their cage. Blood glucose was measured via tail vein 15, 30, and 60 minutes after insulin injection. Mice were provided with 20% glucose solution and removed from ITT if hypoglycemia was observed. Upon completion of ITT, mice were returned to cages with food and water and monitored for adverse effects.

For the intraperitoneal glucose tolerance test, male mice were transferred to new clean cages and fasted for 6 hours prior to GTT. After 5 hours of fasting, the mice were weighed to determine their proper glucose dosage. The appropriate amount of 20% glucose solution was aliquoted for each mouse (2 g/kg). After 6 hours of fasting, the mice had their blood glucose measured via tail vein using a glucometer. This was recorded as the blood glucose at 0-minute timepoint. The mice were then intraperitoneal injected with their aliquoted glucose solution and returned to their cage. Blood glucose was measured via the tail vein 15, 30, 60, 90, and 120 minutes after glucose injection. Upon completion of GTT, the mice were returned to their cages with food and water and were closely monitored to ensure proper recovery.

### BAP Exposure to Hypothalamic Cell Lines.

Hypothalamic neurons from embryonic and adult mice were previously immortalized to generate the clonal cell lines mHypoE-46, mHypoE-44, mHypoE-41, mHypoE-38, mHypoA-59, a FAC-sorted POMC/GFP mouse-derived cell line, mHypoA-POMC/GFP-2, and a mixed culture mHypoA-Bmal1-WT/F cell line (6, 13, 14). The mHypoE-46, mHypoE-44, and mHypoA-Bmal1-WT/F lines were cultured in DMEM containing 1000 mg/L glucose supplemented with 5% FBS and 1% pen/strep. The mHypoE-41, mHypoE-38, and mHypoA-59 cell lines were cultured in DMEM containing 4500 mg/L glucose supplemented with 2% FBS and 1% pen/strep. The mHypoA-POMC/GFP-2 line was cultured in DMEM containing 1000 mg/L glucose supplemented with 2% FBS and 1% pen/strep. All cells were maintained in 5% CO₂ at 37° C. BAP was initially dissolved in DMSO solvent (vehicle control) to achieve 1000x the intended dose. The BAP solution was then diluted 1:1000 with the growth media used for the cell line. For co-treatment experiments, the vehicle control for ATP, UDP, and PKI was water, and for the H89 PKA inhibitor was DMSO. Neuropeptide gene expression levels were determined by qRT-PCR.

### RNA Isolation and RT-qPCR

For cell culture experiments, total RNA was isolated using PureLink RNA isolation kit (Thermofisher Scientific, Burlington, ON, Canada) according to the manufacturer's instruction with on column DNase step (PureLink DNAse kit) or DNase I treatment to remove genomic DNA. For mouse experiments, total RNA was isolated using mirVana PARIS RNA and native protein isolation kit (Thermofisher Scientific). RNA quality and quantity were measured using Nanodrop 2000 and 500-1000 ng of complimentary DNA (cDNA) was synthesized with High Capacity cDNA Reverse Transcription kit (Applied biosystems, Thermofisher Scientific). Quantitative reverse transcription polymerase chain reaction (RT-qPCR) was performed with 12.5 ng of cDNA amplified with Platinum SYBR green qPCR SuperMix-UDG with ROX on an Applied Biosystems Prism 7900HT or PowerTrack Green Master Mix on an Applied Biosystems QuantStudio 5 and gene specific primers as per manufacturer's instructions (Table 1). Data was analyzed using AACT method and normalized to reference gene, either 60S ribosomal protein L7 (Rpl7) or Histone 3a (His).

TABLE 1
List of primer sequences.
GENE PRIMERS

| | | |
|---|---|---|
| RPL7 | F: TCG CAG AGT TGA AGG TGA AG | R: GCC TGT ACT CCT TGT GAT AGT G |
| HIS | F: CGC TTC CAG AGT GCA GCT ATT | R: ATC TTC AAA AAG GCC AAC CAG AT |
| NPY | F: CAG AAA ACG CCC CCA GAA | R: AAA AGT CGG GAG AAC AAG TTT |
| | CAT T | |
| AGRP | F: CGG AGG TGC TAG ATC CAC AGA | R: AGG ACT CGT GCA GCC TTA CAC |
| POMC | F: CCC GCC CAA GGA CAA GCG TT | R: CTG GCC CTT CTT GTG CGC GT |
| P2Y1 | F: GAC ACG AGT TTG TGA AGG CAC | R: CTC AGG GAT GTC TTG TGA CCA |
| | TGT | |
| P2Y2 | F: TGC GCA CCA TTG CCT TGG | R: GGT CAA GTG ATC GGA AGG AGT |
| | AG | |
| P2Y4 | F: AAG ACA GCA TCT TCC CTG CTT | R: ATG CCC CTA TCA ACT GTC ACT |
| P2Y6 | F: TCT TGC ATG AGA CAG ACT CTC CG | R: GCA GTC GCT TGA AAT CCT CAC |
| P2Y12 | F: GGG TCA CAG TGC AAG AAC ACT | R: ATT GGC TGA GGT GGT GTT GA |
| P2Y13 | F: CTC TTC CAA AAC AAA GCT GAT GC | R: GCT CAG ACT TGT TGA AGC CC |
| P2Y14 | F: CCA TGC AAA ATG GAA GTC TG | R: CGG AAA GAC TGG GTG TCT TC |
| P2X1 | F: AAT GGG ACA AAC CGT CGT CA | R: GTC ACG TTC ACC CTC CCC AG |
| P2X2 | F: ATG GGA TTC GAA TTG ACG TT | R: GAT GGT GGG AAT GAG ACT GAA |
| P2X3 | F: CCA TTA AGC AGC CCA CTC CAG | R: GTG CAA GAA AAC CCA CCC CAC |
| P2X4 | F: CTC ATC CGC AGC CGT AAA GT | R: ACG AAC ACC CAC CCA ATG AC |
| P2X5 | F: ATT CCG TCT CAG GGG GAG AA | R: CCG TAG ACA GCG ACC AGT TT |
| P2X6 | F: GGC TAC CAG GAA CGG GAC TT | R: GAA CGG AAG GAT GCT CTG GAC |
| P2X7 | F: AGC ACG AAT TAT GGC ACC GT | R: CCC CAC CCT CTG TGA CAT TCT |
| SESN2 | F: GAA CAA CTC AGG GGG CTT TGA | R: TAT CCA AAT GCG GGG TCC TC |
| TSC2 | F: GAA CTG AGT GGT GAA TGC GG | R: CAA ACG ATC ACC CTG TCC CT |
| ULK1 | F: TCG ACA CCG CGA GAA GCA | R: AAG TCA TAC AGC GCC ACG AT |
| PIK3R1 | F: TAT TGC GAG GGA AGC GAG AC | R: ACT TCG CCG TCT ACC ACT AC |
| RPS6KB1 | F: AGG CGG GAC GGC TTT TAC | R: TCA CAA TGT TCC ATG CCA AGT TC |
| EIF4EBP1 | F: TCA CTA GCC CTA CCA GCG AT | R: ATT GTG ACT CTT CAC CGC CT |
| ATF4 | F: GGA GCA AAA CAA GAC AGC AGC | R: TTG CCT GGA CCT CTT CT |
| EGR1 | F: GCA CCT GAC CAC AGA GTC CTT | R: GCC ACA AAG TGT TGC CAC TG |
| EGR3 | F: TCA GAT GGC TAC AGA GAA TGT G | R: TTC CCA AGT AGG TCA CGG TC |
| CFOS | F: CAA CGA GCC CTC CTC CGA CT | R: TGC CTT CTC TGA CTG CTC ACA |
| CFOS | F: CAA CGA GCC CTC CTC CGA CT | R: TGC CTT CTC TGA CTG CTC ACA |
| NDPK1 | F: TCT TTC CAG TCC GCT GTT GG | R: TTC TGC TCG AAC CGC TTG AT |
| NDPK2 | F: TCT CTT TGC TTC TGT CGC TCA TC | R: CGA TGT AAT GCT GCT TCA GGT |

### Western Blot Analysis

mHypoE-46 cells were serum starved in 5.5 mM glucose (LG) DMEM for 1 hour prior to treatment with vehicle (0.1% DMSO), 100 UM BAP or H20, 500 M 5-Aminoimidazole-4-carboxamide ribonucleotide (AICAR) or 0 mM glucose DMEM. Protein was harvested using 1× cell lysis buffer (Cell Signaling Technologies, Inc, CST) containing protease and phosphatase inhibitors. A total of 30 μg protein was separated on a 12% SDS-polyacrylamide gels and transferred onto PVDF membranes. Membranes were blocked in 5% milk dissolved in Tris-buffered saline with Tween 20 (TBS-T) for 1 h and incubated with primary antibody overnight at 4° C. phospho-AMPKα (Thr172; cat. 2532S, CST), and total AMPKα (cat. 2535S, CST); phospho-p70-S6K (Thr389; cat. 9205S, CST) and total p70-S6K (cat. 9202S, CST); insulin receptor ß (cat. 3025S, CST); phospho-AKT (Ser473; cat. 9271S, CST) and total AKT (cat. 9272S, CST); phospho-ERK1/2 (Thr202/Tyr204; cat. 9101, CST) and total ERK (cat. 4695, CST) were diluted 1:1000 in 5% milk in TBS-T. Membranes were washed and incubated with secondary HRP-linked antirabbit antibody (1:7500 in 5% milk in TBS-T, CST) for 1 h, washed, and imaged using the Signal Fire ECL Reagent (CST) on the iBright 1500 Imaging System (Thermofisher). Protein density was quantified using ImageJ or the iBright Imaging System.

### Results

### Male CD-1 Mice Exposed to BAP in the Drinking Water Lose Weight.

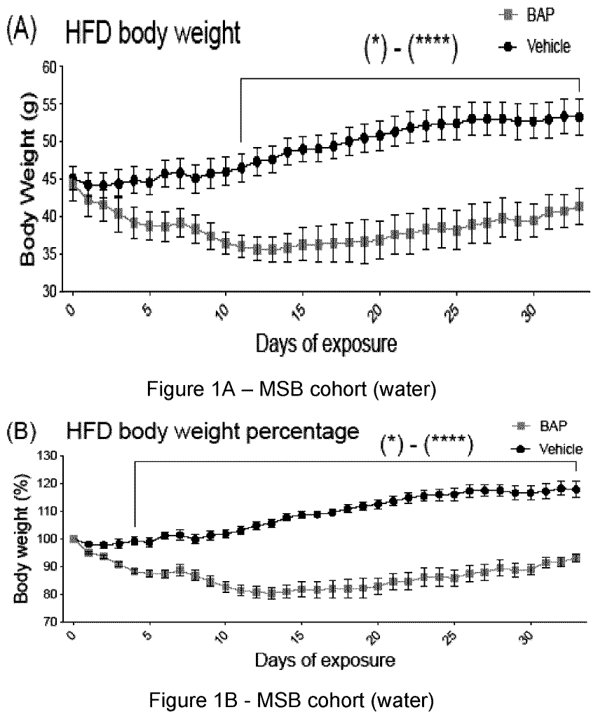

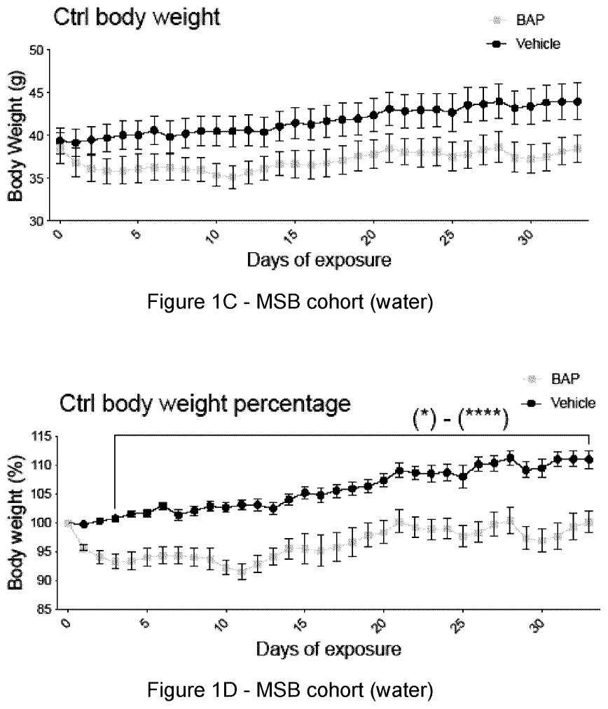

Male CD-1 mice were exposed to BAP continuously via their drinking water for 33 days while on a 60% HFD or sucrose-matched control diet. The male mice that received BAP with a HFD lost a significant amount of body weight, reaching a peak loss of 18% after 13 days of exposure and the effect plateaus from that point onwards (see FIGS. 1A, 1B). The mice on the HFD (no BAP control) continue to gain weight throughout the experimental period weighing 11 grams more than the BAP group at the end of the experimental time period. The male mice on the sucrose-matched control diet that received BAP initially lose weight and achieve peak weight loss of 9% after 12 days of exposure (see FIG. 1C, 1D). After peaking at 12 days, these mice gain back most of the lost weight, but do not return to their starting weight before the end of the experimental period and remain 4 g lighter than the non-BAP exposed control mice. Overall, these results suggest that BAP can reduce body weight in male CD-1 mice regardless of diet.

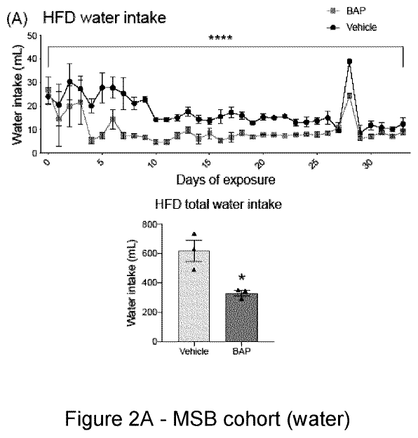

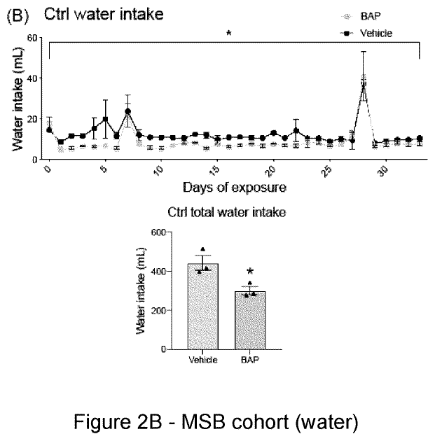

Regardless of diet the mice in the BAP group drink less water when compared to control. The solutions were supplemented with 2% erythritol to overcome a potential taste aversion. With this change in the solution, the mice drank similar amounts of water, although the BAP-exposed mice drank less than controls (see FIGS. 2A, 2B). Thus, we conclude that there is likely a taste aversion to the BAP in the water with and without sweetener. The changes in body weight are unlikely to be strictly due to a reduction in water intake as the mice receiving the control solution and HFD drink less after day 9 of exposure but experience normal weight gain. The spike in water intake on day 28 was the result of stress from fasting for the insulin tolerance testing.

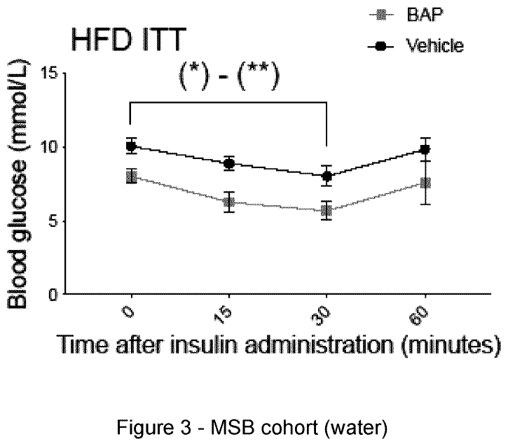

After 33 days on the HFD with continuous BAP exposure, insulin sensitivity was tested in the mice. One week prior to sacrificing, the mice were fasted for 4 hr and underwent an intraperitoneal insulin tolerance test (ITT) to assess changes in insulin sensitivity. The response to insulin across all treatment groups was impaired with HFD (see FIG. 3). Mice on a HFD receiving BAP had significantly lower fasting blood glucose levels when compared mice on a HFD without BAP (see FIG. 3). The ITT results suggest that BAP may in part reduce body weight by improving glucose homeostasis or vice versa.

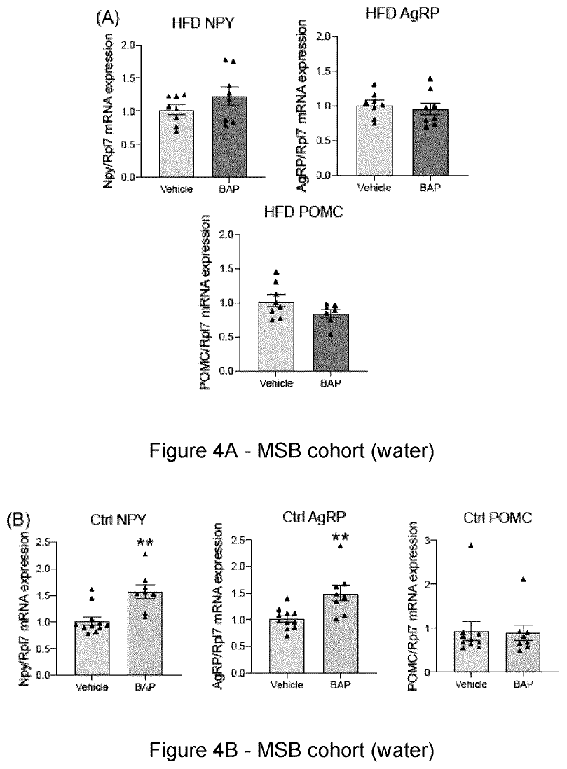

After sacrificing the mice, their hypothalamic were collected for gene expression analysis. RNA was isolated from the hypothalamic and gene expression was quantified using qRT-PCR. The mice on a HFD receiving BAP did not have any significant changes in any of feeding-related neuropeptides measured when compared to its control (see FIG. 4A). Whereas the mice on a sucrose-matched control diet receiving BAP have significantly higher hypothalamic expression of orexigenic feeding neuropeptides Npy and AgRP when compared to their controls (see FIG. 4B). There is no change in Pomc mRNA expression with BAP. This would indicate a compensatory dysregulation of neuropeptides due to the weight loss.

### Female CD-1 Mice Exposed to BAP in the Drinking Water Lose Weight.

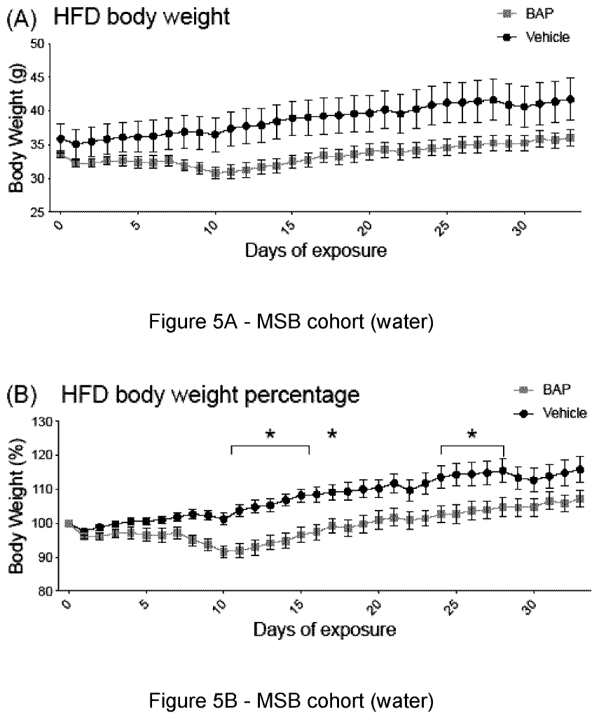

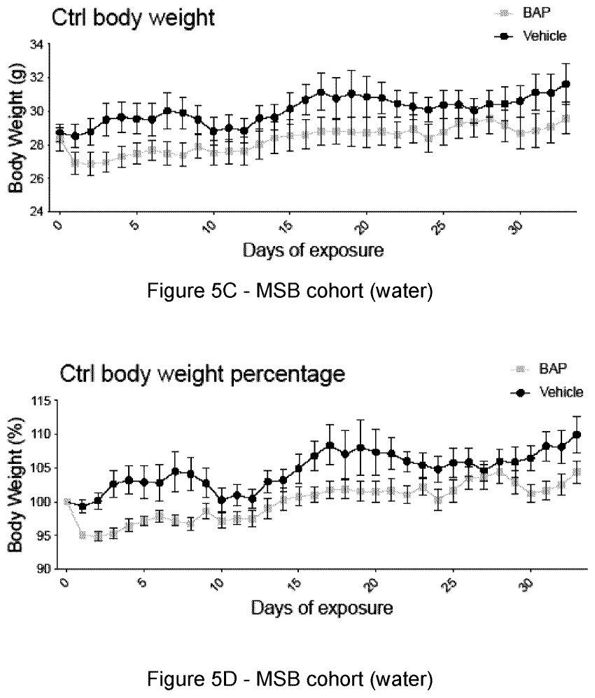

Female mice were exposed to BAP continuously via their drinking water for 33 days while on a 60% HFD or sucrose-matched control diet, using the same paradigm as the male group. The female mice on a HFD receiving BAP initially lose 9% of their body weight after 9 days of exposure but return to their starting weight after 34 days of exposure (see FIGS. 5A, 5B). Even though they return to their starting body weight before the end of the experiment, they still weigh 3 g less than the control group receiving the same diet. The mice on the control diet receiving BAP initially lose 5% of their body weight after 3 days of exposure but it is quickly regained and these mice return to their starting weight (see FIGS. 5C, 5D). Overall, the body weight results in the female CD-1 mice suggest that BAP can significantly reduce body weight but not to the same extent seen in male CD-1 mice when in the drinking water.

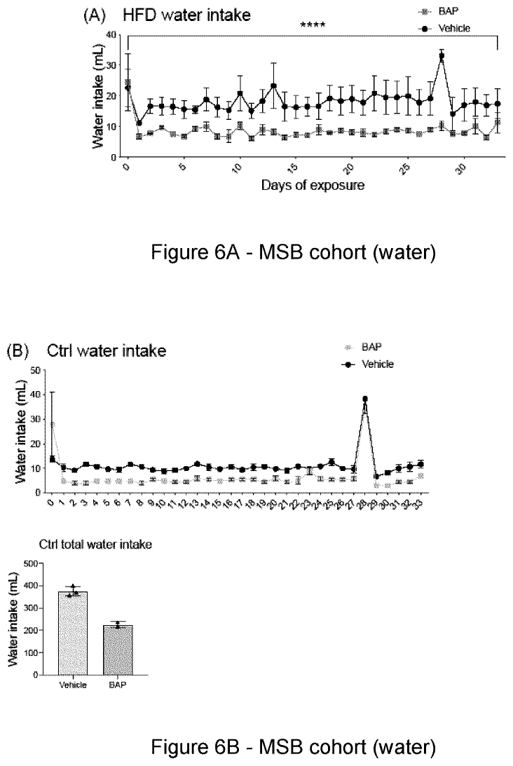

Similar to the male mice, the female mice receiving BAP, regardless of diet, drink significantly less of the solution, even with the erythritol sweetener supplement (see FIGS. 6A, 6B). Furthermore, the elevated water intake on day 28 was the result of stress from fasting for the insulin tolerance testing.

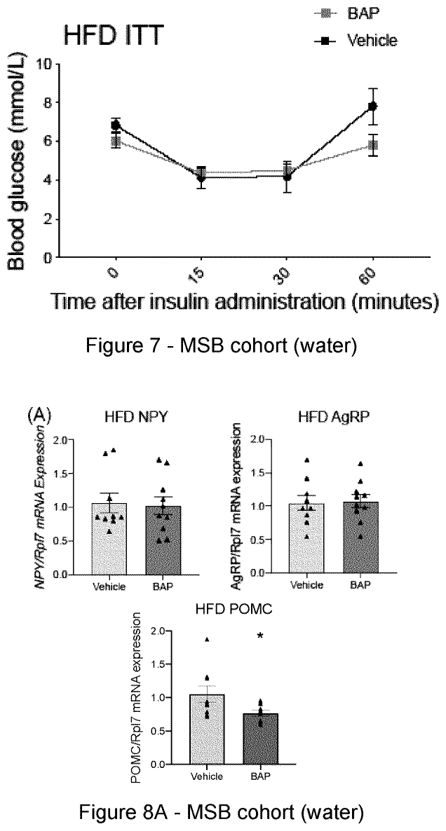

One week prior to sacrificing, the female mice were fasted for 4 hr and underwent an ITT to assess insulin sensitivity. All groups show a similar strong response to insulin and unlike the male CD-1 mice, there are no significant differences in fasting blood glucose with BAP (see FIG. 7).

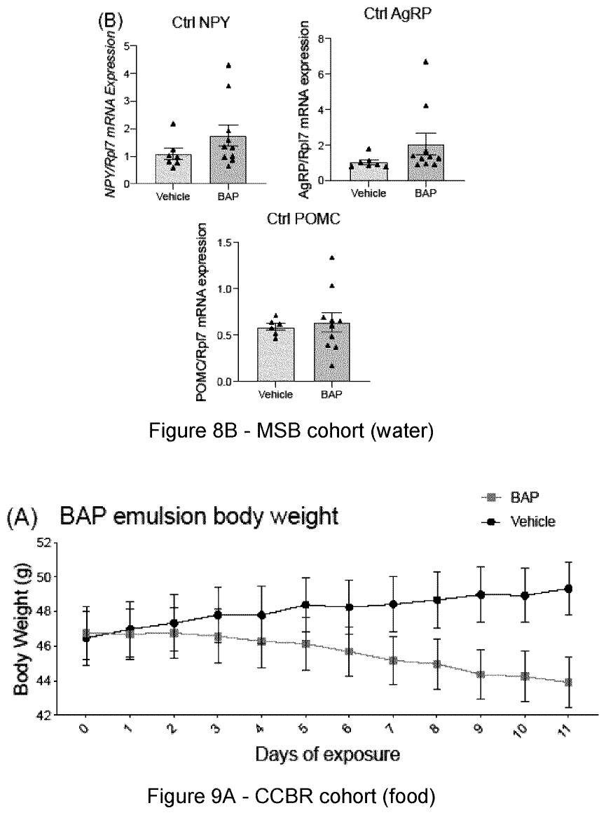

After sacrificing the mice at the end of the exposure period, their hypothalamicre collected for gene expression analysis. The female mice on a HFD and receiving BAP have decreased mRNA expression of Pomc but no significant changes in Npy or Agrp (see FIG. 8A). The female mice on the control diet receiving BAP trended towards increases in Npy and AgRP mRNA expression, but overall do not have significant changes in the feeding neuropeptides measured (see FIG. 8B).

### Male CD-1 Mice Fed a BAP Emulsion Lose Weight.

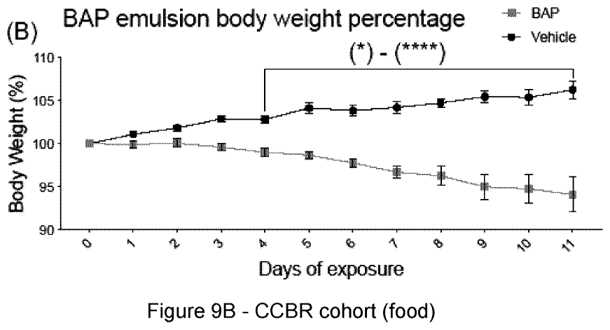

Since the BAP in the water was consumed at lower levels, possibly due to a taste eversion, the BAP was then added to a honey emulsion and fed orally to the mice. Male CD-1 mice were exposed to BAP daily at a dosage of 300 mg/kg/day via an emulsion with carboxymethyl cellulose and honey while on a 60% HFD. The mice exposed to BAP lost 5% of their body weight after 11 days of BAP exposure and weighed 6 grams (11%) less than their control counterparts (see FIGS. 9A, 9B), again demonstrating the weight loss potential of BAP in mice.

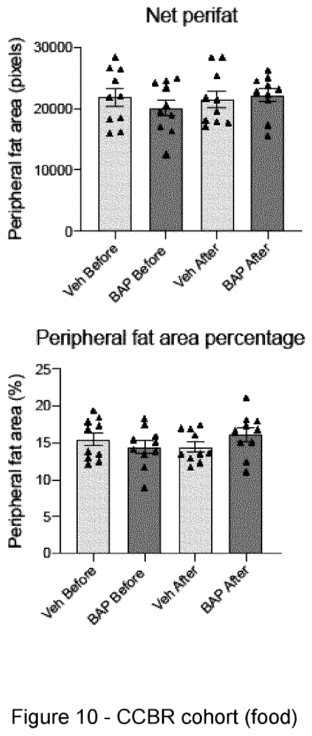

This weight loss was not reflected in changes in peripheral fat mass, assessed via Bruker scanning at 11 days, as we do not observe any significant changes in net peripheral fat nor peripheral fat mass percentage (see FIG. 10). The lack of significant changes in fat mass as measured by the Bruker scan may be the result of insufficient sensitivity of the Bruker scanning method and would require a longer testing period.

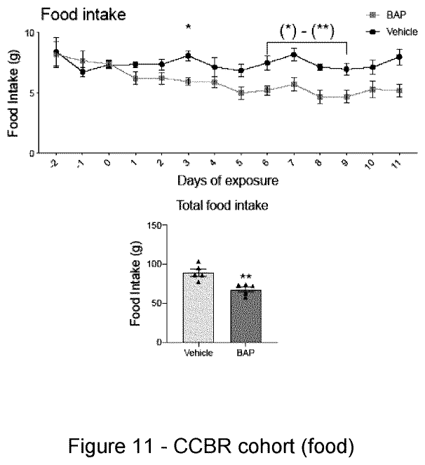

The changes in body weight seem to be primarily caused by a reduction in food intake, as mice receiving BAP eat 22% less HFD when compared to the control group over the exposure period (see FIGS. 11A, 11B). Overall, these results suggest that BAP is able to reduce weight in male CD-1 mice while on a HFD and the cause seems to be a reduction in food intake from these preliminary results.

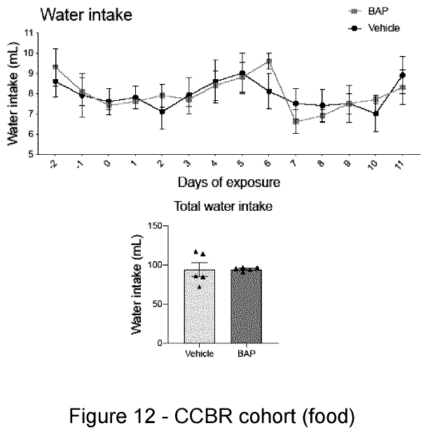

The water intake of these mice is unaffected by BAP throughout the exposure period (see FIG. 12). Since the reduction in water intake was not observed in the male CD-1 mice receiving the BAP emulsion, the reduction in water intake in the first set of experiments (FIG. 1) was primarily the result of the administration method, and a taste aversion to BAP in the water, rather than the effects of BAP itself.

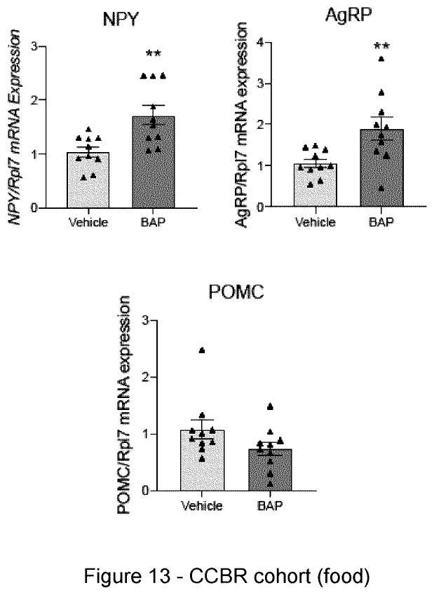

The mice were fed their respective emulsion 2 hr prior to sacrificing and tissue collection, to assess early changes in hypothalamic gene expression induced by BAP. The hypothalamic mRNA expression of orexigenic feeding neuropeptides Npy and Agrp are significantly increased by BAP, whereas, there are no significant changes in the mRNA expression of the anorexigenic feeding neuropeptide Pomc (see FIG. 13). Again, the cause of the weight loss appears to be due to a reduction in food intake, but it is not immediately apparent that the feeding-related neuropeptides studied are playing a significant role in the short-term. If anything, the changes in the neuropeptide mRNA levels are opposite to that expected, and therefore may be playing a compensatory role in the hypothalamus due to the weight reduction, as would be expected if leptin levels were decreased. However, other neuropeptides involved in feeding regulation will also be studied.

### Male CD-1 Mice Fed a BAP Emulsion Lose Weight—Replicate at BSF.

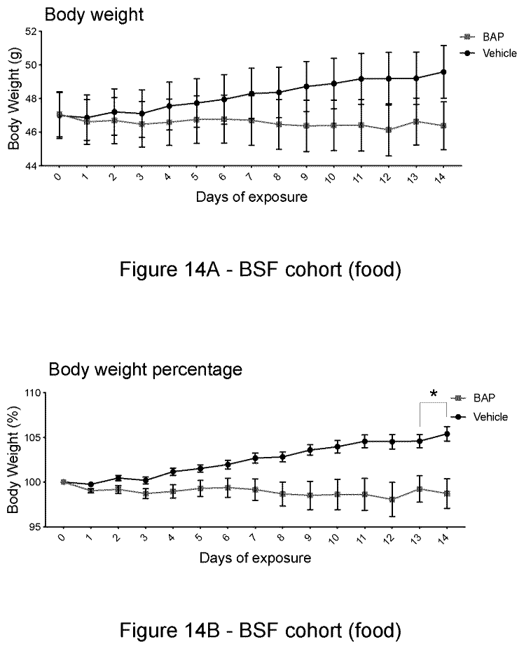

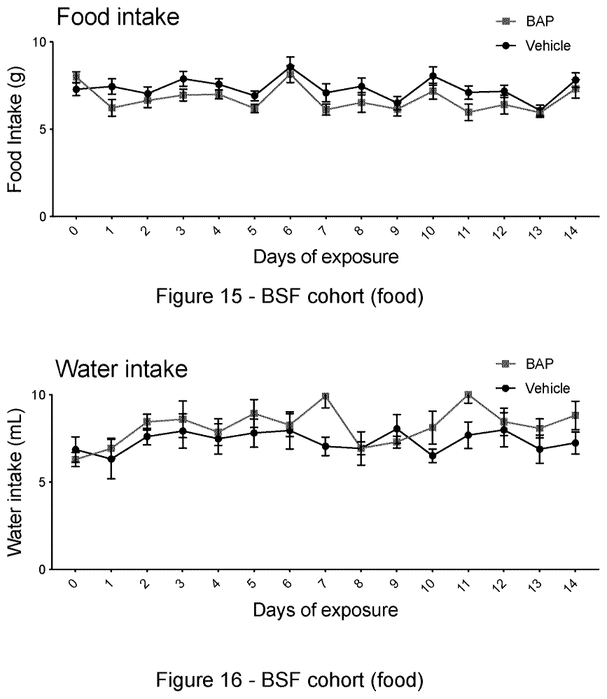

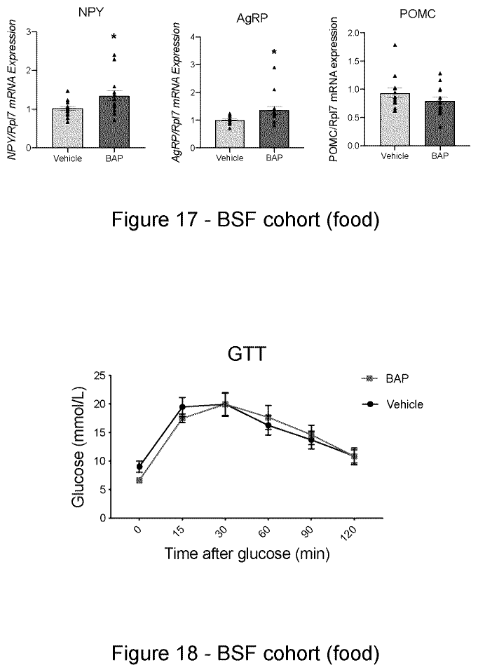

The BAP emulsion experiment was replicated in a second animal facility. Male mice were again exposed to BAP daily at a dosage of 300 mg/kg/day via an emulsion with carboxymethyl cellulose and honey while on a 60% HFD. After 4 weeks on the HFD, the mice exposed to BAP for 14 days lost 1.3% of their body weight while continuing on the HFD and weighed 3.2 grams (6.46%) less than their control counterparts (see FIGS. 14A, 14B), again demonstrating the weight loss potential of BAP in mice. The changes in body weight were not reflected by a reduction in daily average food intake, as mice receiving BAP did not eat significantly less HFD when compared to the control group over the exposure period (see FIG. 15). There was no change in water intake (see FIG. 16). Again, paradoxically, there was a modest increase in Npy and Agrp, as previously, likely due to a counteractive effect in response to the weight loss, but no change in Pomc (see FIG. 17). There was also no change in glucose uptake in a GTT (see FIG. 18).

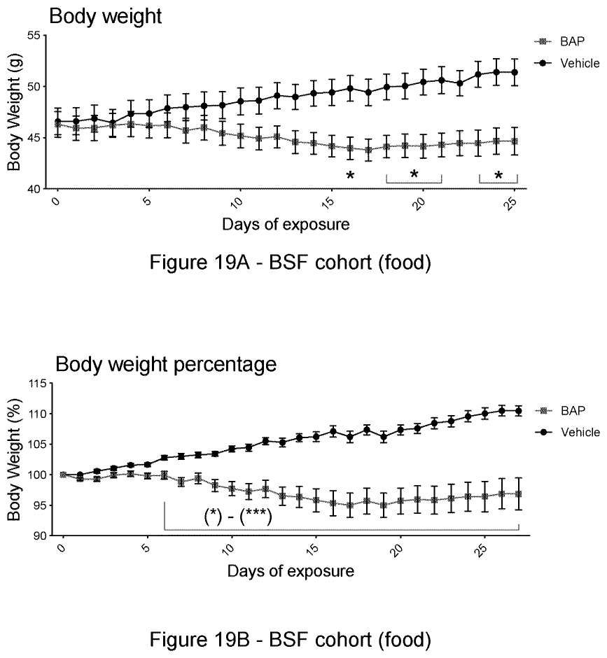

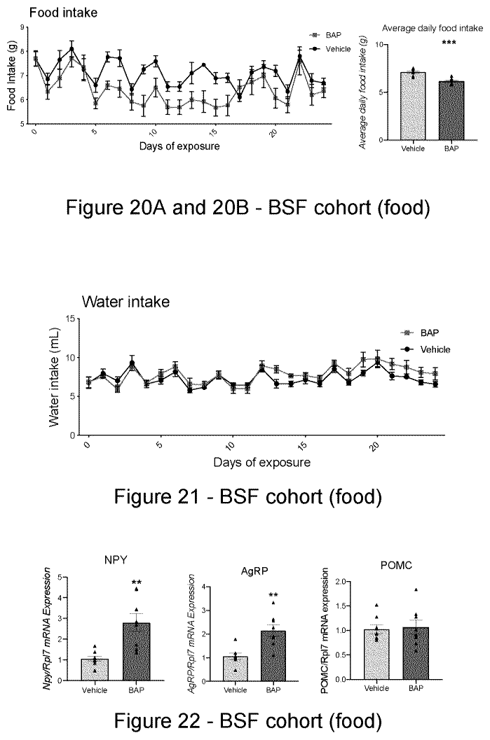

These experiments were continued for 28 days in another cohort of male mice using the same paradigm. The mice exposed to BAP lost 5% of their body weight after 28 days of BAP exposure and weighed 6.73 grams (13%) less than their control counterparts (see FIGS. 19A, 19B). The changes in body weight were reflected by a mild reduction in daily average food intake (13%), as mice receiving BAP ate significantly less HFD when compared to the control group over the exposure period (see FIGS. 20A, 20B). There was no change in water intake (see FIG. 21). Again, paradoxically, there was a modest increase in Npy and Agrp, as previously, likely due to a counteractive effect in response to the weight loss, but no change in Pomc (see FIG. 22).

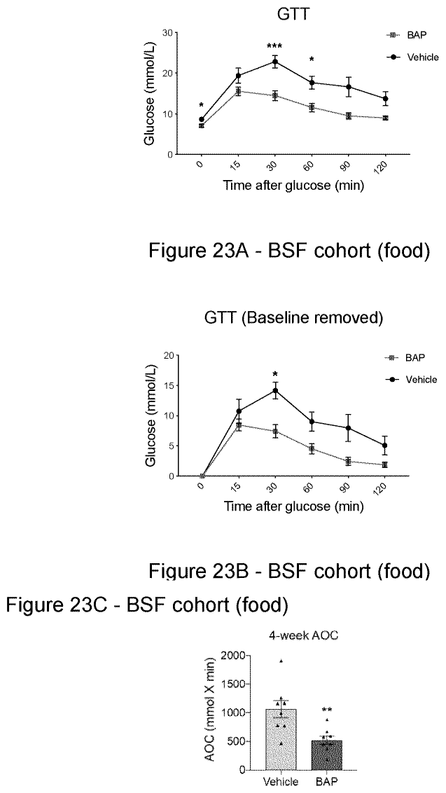

Mice on the HFD had an impaired glucose response, while the mice with the BAP had an improved glucose response (see FIGS. 23A, 23B). Mice had significantly lower fasting blood glucose levels when compared mice on a HFD without BAP (see FIG. 23C). Again, the GTT results suggest that BAP may in part reduce improve glucose homeostasis with the loss of body weight.

Using this same paradigm, the male mice were exposed to three increasing doses of BAP, 75, 150, and 300 mg/kg/day via an emulsion with carboxymethyl cellulose and honey while on a 60% HFD for 11 days and weight and food intake was measured. The mice exposed to 150 and 300 mg/kg/day

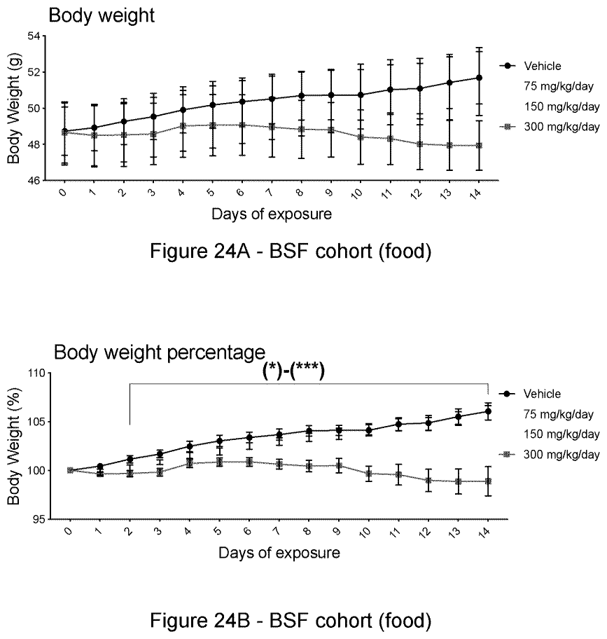

BAP for 14 days did not gain weight on the HFD and lost 1.1% of their body weight, respectively, while continuing on the HFD, and weighed 2.16 (4.2%) and 3.7 grams (7.2%) less than their control counterparts, respectively (see FIGS. 24A, 24B). There was no change at the 75 mg/kg/day dose.

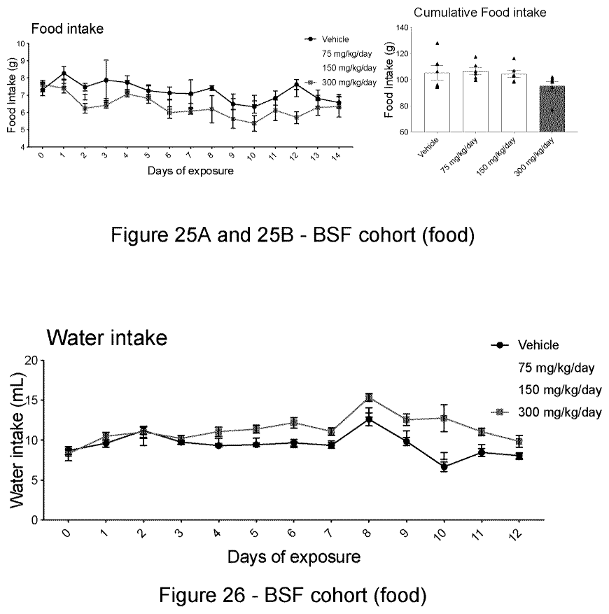

There was a trend towards a decrease in food intake that did not reach significance in the 300 mg/kg/day BAP after 14 days of exposure (see FIGS. 25A, 25B). However, this was significant by an unpaired Student's t-test at 8 days onward. There was no change in water intake (see FIG. 26). No ITT or GTT was done in this cohort to evaluate neuropeptides without the added stress of the procedure.

### Female CD-1 Mice Fed a BAP Emulsion Repeated Due to Stress Issues at the CCBR and Maintain Their Weight on a HFD at the BSF.

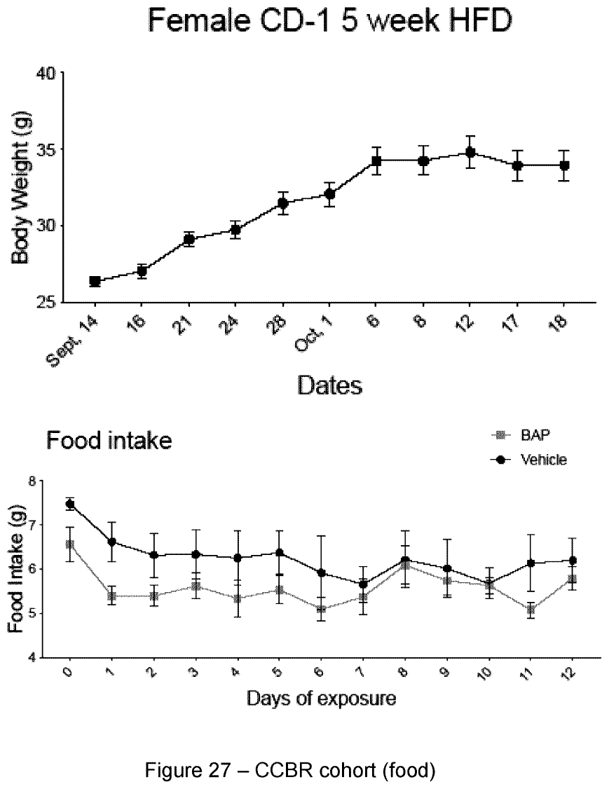

The experiment with the female mice had to be repeated due to apparent stress in the mice causing them to stop gaining weight on a HFD after 3 weeks. This is likely due to the ongoing construction at the MSB/CCBR site, as well as excessive activity due to moving of the animals from the MSB to the CCBR animal facility. Female CD-1 mice were exposed to BAP daily at a dosage of 300 mg/kg/day via an emulsion with carboxymethyl cellulose and honey while on a 60% HFD. The female mice stopped gaining weight on the 60% HFD after only 3 weeks, and even before the initiation of the BAP emulsion feeding (see FIG. 27). The BAP group initially seem to consume less food and water than the control group, but this effect appears to be independent of BAP, as the BAP group was already consuming less than the control group prior to any BAP exposure (see FIG. 27). As with the male CD-1 cohort, there was no significant difference in the water intake with BAP emulsion (data not shown). The mice were fed their respective emulsion 16 hr prior to sacrificing and tissue collection. There were no significant changes in the hypothalamic expression of any of the feeding neuropeptides measured (data not shown). Since there was no issue with weight gain in our first set of experiments (see FIG. 5A), other confounding factors appear to be the cause of the problem. These experiments were repeated in our new animal location in the Biological Sciences Facility (BSF) when allowed due to the pandemic restrictions.

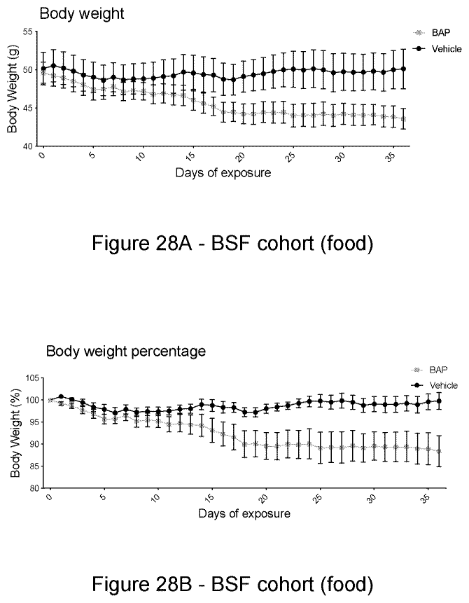

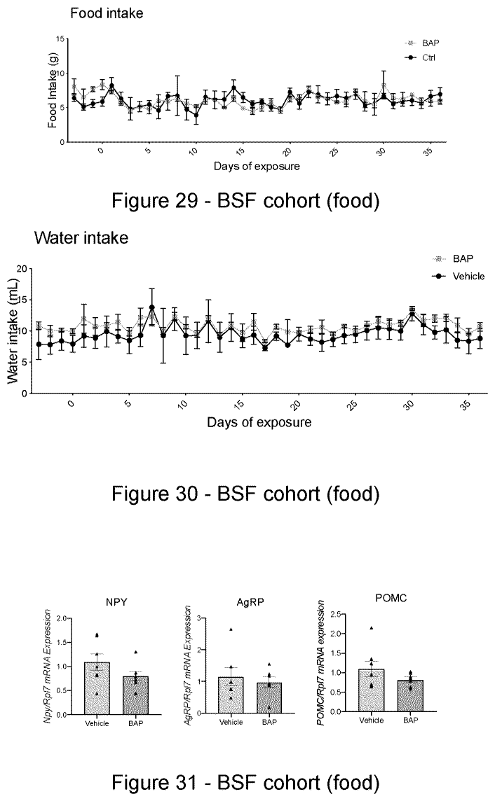

Because female mice take more time to develop insulin resistance, we decided to expose the next cohort of female mice to 16 weeks of 60% HFD followed by 36 days of BAP daily at a dosage of 300 mg/kg/day via an emulsion with carboxymethyl cellulose and honey while on a 60% HFD. This experiment was undertaken in another animal facility (BSF). Two weeks prior to start of BAP administration, the mice were fasted for 6 hours and underwent an ipITT. All mice displayed a similar strong response to insulin administration and most of the mice became hypoglycemic and were subsequently rescued with glucose injection. Therefore, even with the longer 16 week 60% HFD period prior to BAP exposure, the female mice remain insulin sensitive. The mice exposed to BAP did not gain weight on a HFD and weighed 6.53 grams (13%) less than their control counterparts (see FIGS. 28A, 28B). The changes in body weight were not reflected by a reduction in food intake, as mice receiving BAP did not eat significantly less HFD when compared to the control group over the exposure period (see FIG. 29). There was no change in water intake (see FIGS. 30).

After sacrificing the mice at the end of the exposure period, their hypothalamus were collected for gene expression analysis. Again, there were no changes in Npy, Agrp, and Pomc, as previously, likely reflecting a lack of food intake changes (see FIG. 31).

One week prior to sacrificing, the female mice were fasted for 4 hr and underwent an ITT to assess insulin sensitivity. All groups show a similar strong response to insulin, indicating a continuing sensitivity to insulin despite the long-term exposure to the 60% HFD, and unlike the male CD-1 mice, there are no significant differences in fasting blood glucose with BAP (data not shown). A number of the female mice experienced hypoglycemic events requiring glucose rescue despite decreasing the insulin dose for the ITT.

BAP Induces Anorexigenic Neuropeptide Expression in Primary Culture from CD-1 Mice.

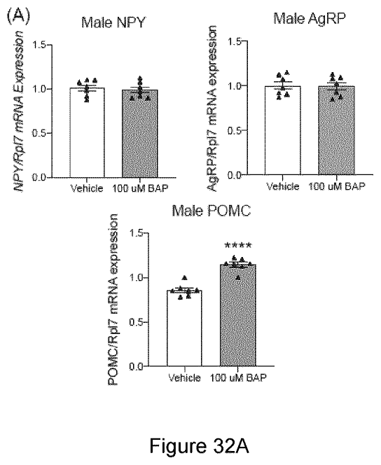

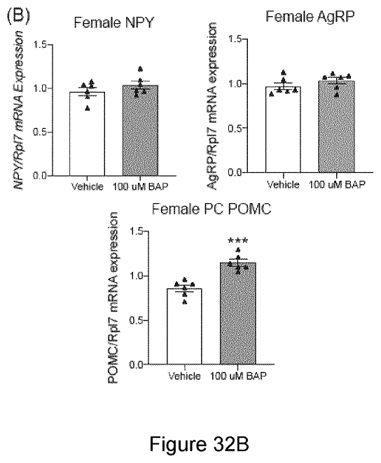

Because the BAP was being fed to the mice, it is also important to determine what are the direct effects of BAP on native hypothalamic neurons, since the results may be due to an indirect effect from the periphery. Hypothalamus were extracted from male and female CD-1 mice fed a normal chow diet, and primary hypothalamic neurons were cultured from individual mice. The neurons were treated with 100 UM of BAP for 16 hr to assess the effect of BAP on feeding-related neuropeptide expression. In both sexes, BAP had no effects on the expression of Npy or Agrp, but was able to significantly increase the expression of Pomc (see FIGS. 32A, 32B), indicating that BAP may induce an anorexigenic response through the enhanced expression of this neuropeptide.

### BAP has Significant Effects on Neuropeptide Expression in Hypothalamic Cell Lines.

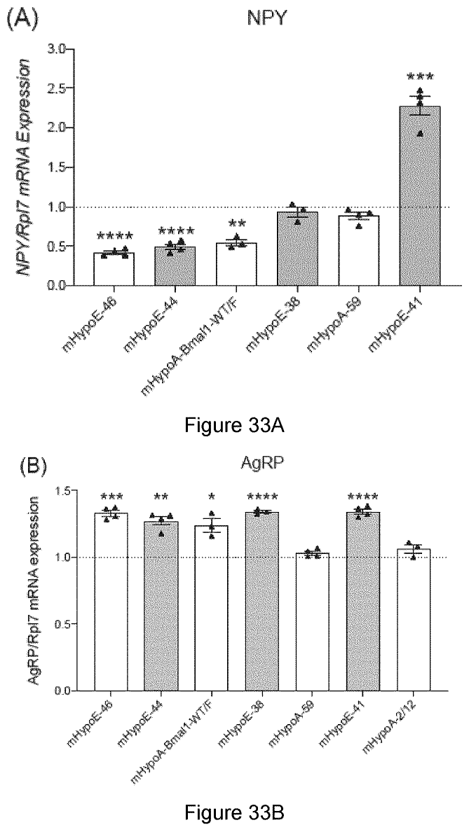

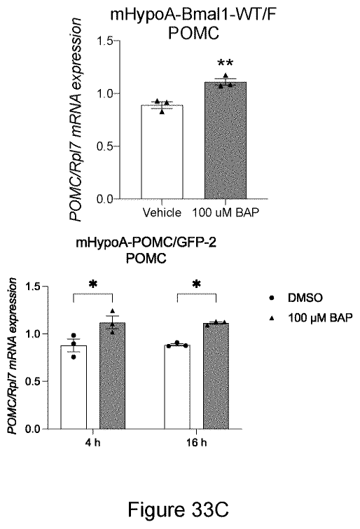

To test the effects of BAP directly on defined hypothalamic neurons, we treated multiple murine-derived Npy-expressing cell lines for 16 hr with 100 μM BAP. The Npy-expressing lines include male-derived embryonic lines, mHypoE-46, mHypoE-44, and mHypoE-38. Also included were female-derived adult-and embryonic-derived lines, mHypoA-59 and mHypoE-41, respectively, as well as, a heterogeneous population of hypothalamic neurons derived from a female whole mouse hypothalamus, mHypoA-Bmal1-WT/F. BAP supressed mRNA expression of Npy in the mHypoE-46, mHypoE-44, and mHypoA-Bmal1-WT/F cell lines, but increased Npy expression in the mHypoE-41 cell line. BAP had no effect in the mHypoA-59 or mHypoE-38 lines (see FIG. 33A), whereas Agrp mRNA expression was increased in all of cell lines tested, except for the mHypoA-59 cell line (see FIG. 33B). On the other hand, Pomc expression was significantly induced in the mHypoA-Bmal1-WT/F and mHypoA-POMC/GFP-2 cell lines (see FIG. 33C). Overall, we observe that BAP can affect the expression of orexigenic Npy and Agrp, and anorexigenic Pomc, feeding neuropeptides in multiple neuronal models, that would dictate an overall anorexigenic response. We studied the mHypoE-46 and mHypoE-44 cell lines further, as we observed the strongest Npy repression in these cell lines, and also the mHypoE-41 cell line, which was the only cell line where we observed a BAP-mediated induction of Npy gene expression.

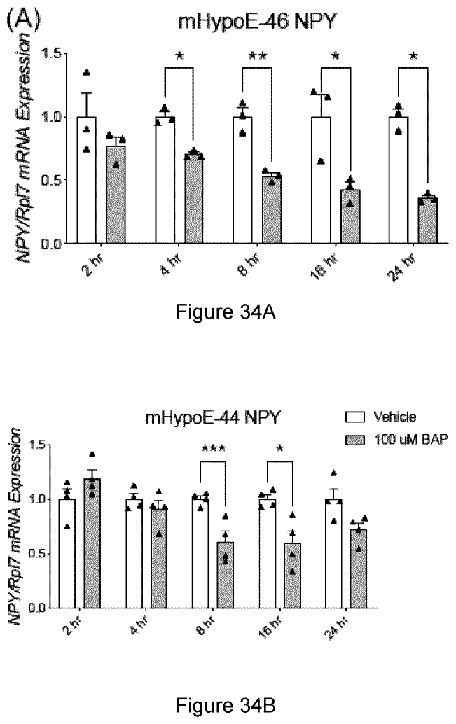

The mHypoE-46 and mHypoE-44 cell lines were treated with 100 μM of BAP for 2, 4, 8, 16, and 24 hr. In the mHypoE-46 cell line, BAP was able to significantly downregulate Npy mRNA expression at all of the time points tested (see FIG. 34A). In the mHypoE-44 line, BAP was able to significantly downregulate the mRNA expression of Npy at 8 and 16 hr (see FIG. 34B).

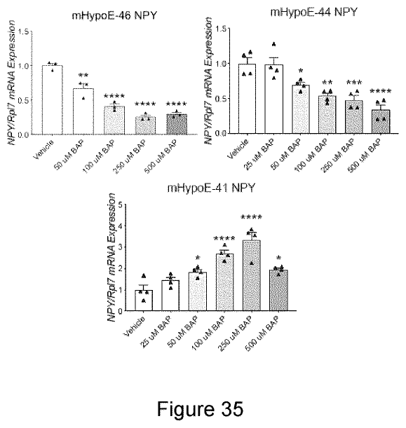

The mHypoE-46, mHypoE-44, and mHypoE-41 cell lines were treated with increasing concentrations of BAP for 16 hr. In the mHypoE-46 line, BAP was able to significantly decrease Npy mRNA expression with as little as 50 μM and the magnitude of the downregulation plateauing at the 250 μM dose (FIG. 35). In the mHypoE-44 line, BAP decreases Npy in a dose dependent manner with the strongest effects observed at the 500 μM dose (see FIG. 35). In the mHypoE-41 line, BAP has the opposite effect, wherein it increases the expression of Npy (FIG. 35). The strongest effect is observed with the 250 μM dose and becomes attentuated at the 500 μM dose. Overall, the magnitude of the effects of BAP increase with concentration until the 500 μM threshold.

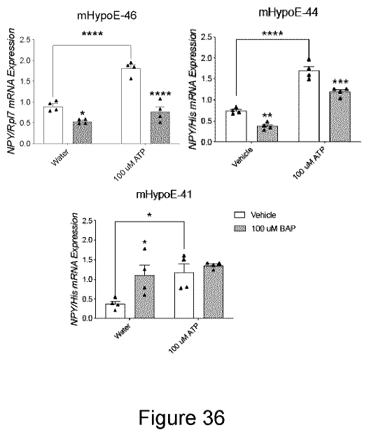

The mHypoE-46, mHypoE-44, and mHypoE-41 cell lines were co-treated with 100 μM ATP, the endogenous agonist for most of the P2 purine receptors, and 100 μM BAP for 16 hr. In the mHypoE-46 line, ATP induces the expression of Npy, but BAP is able to overcome this induction and supress Npy mRNA levels comparable to control/BAP group (see FIG. 36). In the mHypoE-44 line, ATP induces the expression of Npy, but BAP is still able to overcome this induction (see FIG. 36). This indicates that the effect of BAP in the mHypoE-46 and mHypoE-44 lines is likely independent of signaling by ATP. In the mHypoE-41 line, ATP induces the expression of Npy, but BAP is unable to further induce Npy beyond this point (see FIG. 36). This suggests that the effect of BAP in the mHypoE-41 cell line may be dependent on ATP signaling or a plateau in the expression of Npy has been reached.

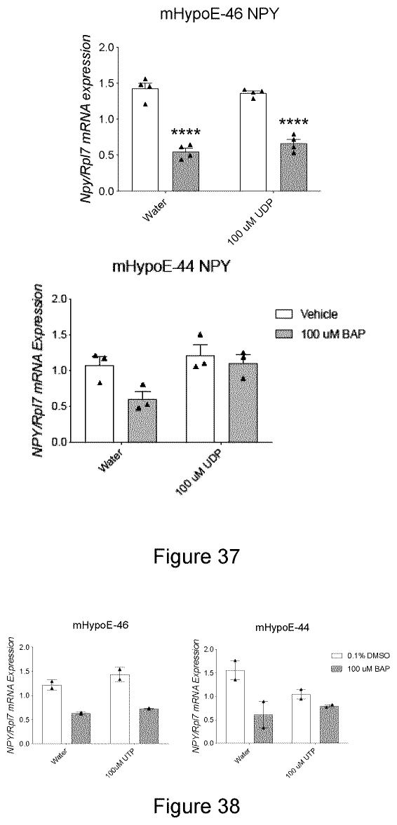

The mHypoE-46 and mHypoE-44 cell lines were co-treated with 100 μM UDP, the endogenous agonist for the P2Y6 receptor, and 100 μM BAP for 16 hr. In the mHypoE-46 line, UDP has no effect on the expression of Npy and BAP is still able to supress the gene (FIG. 37). As such, the effect of BAP in the mHypoE-46 line is independent of UDP signaling. In the mHypoE-44 line, UDP is again unable to affect the expression of Npy, but is able to block the repression of Npy levels by BAP (see FIG. 37). This suggests that the effect of BAP in the mHypoE-44 line is potentially dependent on UDP signaling and its receptor P2Y6. A caveat to this result is that the supression of Npy mRNA levels by BAP in the mHypoE-44 neurons in the vehicle control is not significant by a two-way ANOVA analysis, but is significant by an unpaired Student's t-test.

The mHypoE-46 and mHypoE-44 cell lines were co-treated with 100 μM UTP, the endogenous agonist for the P2Y4 receptor, and 100 μM BAP for 16 hr. In the mHypoE-46 line, UTP has no effect on the expression of Npy and BAP is still able to supress the gene (FIG. 38). As such, the effect of BAP in the mHypoE-46 line is independent of UTP signaling. In the mHypoE-44 line, UTP may decrease the basal expression of Npy, and also appears to be able to block the repression of Npy levels by BAP (see FIG. 38). However, with an n=2 these experiments will need to be replicated. This suggests that the effect of BAP in the mHypoE-44 line is potentially partially dependent on UTP signaling and its receptor P2Y4. The potential involvement of the P2Y receptors in the mHypoE-44 will be studied further.

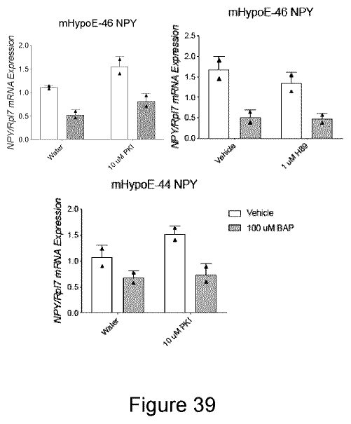

Previous studies have implicated the role of protein kinase A (PKA) in the effects of BAP. To assess this in our neurons, we cotreated the mHypoE-46 and mHypoE-44 with 10 μM PKI or 1 μM H89, both of which are commonly used PKA inhibitors, with 100 μM BAP for 16 hr. In both cell lines, inhibition of PKA with either inhibitor is unable to block the effects of BAP on Npy expression (see FIG. 39). Although PKA has been shown to be crucial for the effect of BAP on melanogensis in B16 melanoma cells, it does not appear to be involved in the effects of BAP in hypothalamic neurons from these preliminary results.

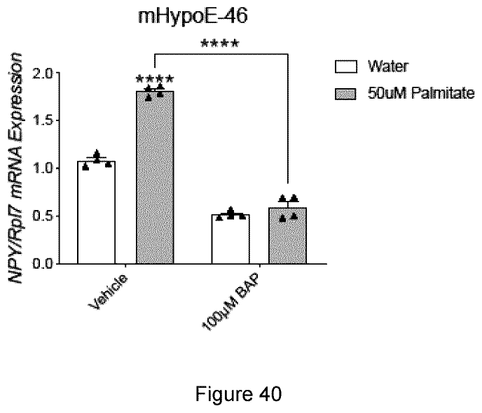

Previous studies from our laboratory have demonstrated the detrimental effects of palmitate, the most common saturated fatty acid in our diet in a number of our hypothalamic cell lines (15). These findings include the significant induction of Npy expression (16, 17). To assess whether BAP can block the effects of palmitate, due to its potential anti-inflammatory properties, we cotreated the mHypoE-46 neurons with 100 μM BAP and 50 μM palmitate for 16 hr. We observed that palmitate induced a nearly two-fold increase in Npy mRNA levels, and BAP rescues the palmitate-mediated increase in Npy expression (see FIG. 40). This suggests that BAP can be beneficial by supressing Npy expression and potentially blocking the stimulatory effects of palmitate on Npy gene expression.

BAP Decreases AMPK Activation in the mHypoE-46 Hypothalamic Cell Line.

One potential signal transduction pathway that has been linked to energy homeostasis and feeding behaviour is the AMP-kinase pathway (AMPK). Activation or phosphorylation of AMPK is an indication of low energy levels in the cell, thus when nutrients are low AMPK will be phosphorylated leading to an increase in neuropeptides related to feeding, such as NPY. Therefore, since we demonstrate a decrease in feeding in our mice, we would expect a decrease in

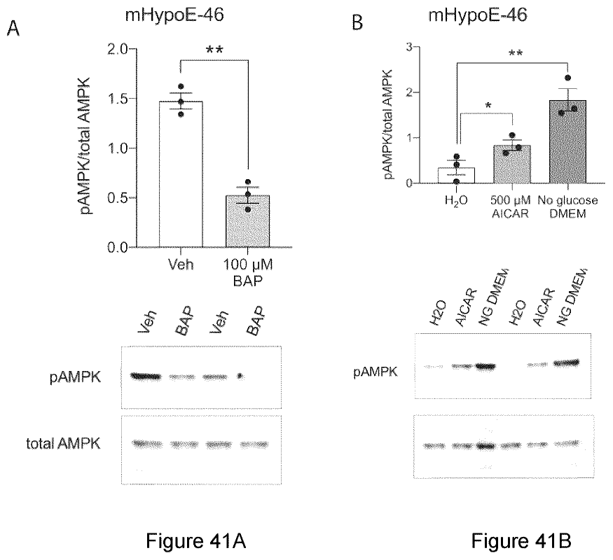

AMPK phosphorylation, mimicking our decrease in Npy gene expression. Treatment of the mHypoE-46 cell line with 100 μM BAP results in a significant decrease in AMPK phosphorylation or activation (see FIG. 41A). On the other hand, the positive controls, the AMPK activator AICAR and addition of cell culture medium without glucose (low nutrient levels) significantly activate AMPK as expected (see FIG. 41B). This indicates that BAP may, in part, act through the AMPK signal transduction pathway to alter feeding neuropeptide levels to ultimately result in a decrease in appetite, food intake, and weight.

BAP Regulates a Number of Genes Involved in mTORC1 Signal Transduction in the mHypoE-46 Hypothalamic Cell Line.

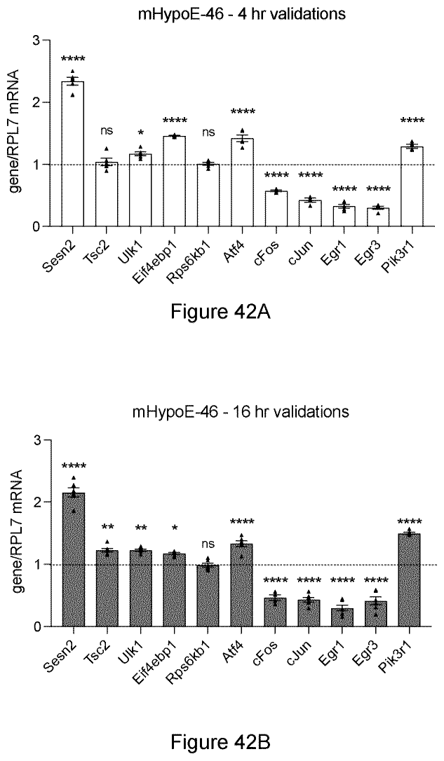

To assess global mRNA gene expression changes, we exposed the mHypoE-46 line to 100 μM BAP for 4 and 16 h and assess mRNA expression via RNA-sequencing (RNA-seq). One of the more highly induced signal transduction pathway was related to the core component mTORC1. It is already known that a decrease in mTORC1 signaling results in a decrease in Npy through decreases in p70-S6K activity (18-20). Our RNAseq analysis was validated in the mHypoE-46 cell line, wherein we found that a number of genes were altered by BAP exposure in the neurons. Specifically, the gene expression of the upstream inhibitors of mTORC1, sestrin2 (Sesn2) (21) and tuberous sclerosis complex 2 (Tsc2) were both increased with BAP; whereas the gene expression of two downstream effectors inhibited by mTORC1, 4e-BP1 (Eif4ebp1) and ULK1 (Ulk1), were increased as well, indicating an overall negative regulation of mTORC1 activity (see FIG. 42A and 42B).

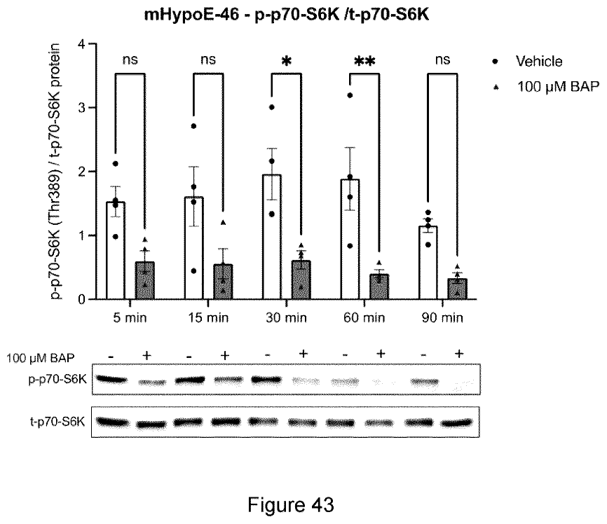

The mHypoE-46 neurons were serum-starved in plain, low-glucose (5.5 mM) DMEM for 1 hour to eliminate basal activation from the media. The neurons were then exposed to 100 μM BAP for 5, 15, 30, 60 and 90 minutes followed by protein isolation and Western blot analysis to assess phosphorylation of p70-S6K (Thr389), a downstream kinase whose activity is increased by mTORC1. Importantly, p70-S6K phosphorylation (activity) as assessed by Western blot analysis was significantly downregulated by BAP, which could ultimately lead to a repression of the Npy gene (see FIG. 43). A summary of the mTORC1 pathway analysis is listed in Table 2.

TABLE 2
qRT-PCR validation of the RNAseq analysis
for genes involved in mTORC1 signaling.

| | | |
|---|---|---|
| Protein | RNA-seq data | [symbol] |

| (Gene) | 4 h | 16 h | [symbol] | [symbol] |
|---|---|---|---|---|
| Sestrin2 | ↑ | ↑ | ↑ | ↑ |
| (Sesn2) | | | | |
| TSC2 | ns | ↑ | ns | ↑ |
| (Tsc2) | | | | |
| S6K | ns | ↓ | ns | ns |
| (Rps6kbI) | | | | |
| 4e-BPI | ↑ | ↑ | ↑ | ↑ |
| (Eif4ebp I) | | | | |
| ULK I | ns | ↑ | ↑ | ↑ |
| (Ulk I) | | | | |

[symbol]

indicates data missing or illegible when filed

Finally, a number of transcription factors that could potentially bind to the 5′ regulatory region of the NPY gene to regulate its transcription were found to be altered by BAP in the RNAseq analysis. Two strong candidates that have been shown to control Npy expression (22, 23), the early growth response protein 1 and 3 (Egr1/3) were highly downregulated genes by BAP at both 4 and 16 h. Similarly, the components of the AP1 binding complex, c-fos and c-jun were both significantly downregulated by BAP. There are also a number of activating AP1 binding sites in the Npy 5′ regulatory region (24), thus a decrease in these transcription factors could also lead to a downregulation of NPY. We validated these results in the mHypoE-46 cell line (see FIGS. 42A and 42B), and predict that a repression of these transcription factors by BAP would result in decreased activation of the Npy gene and results in decreased NPY levels.

BAP Represses the Mitogen-Activated Protein Kinase (MAPK) Signal Transduction Pathway Through a Decrease ERK1/2 Phosphorylation in the mHypoE-46 Hypothalamic Cell Line.

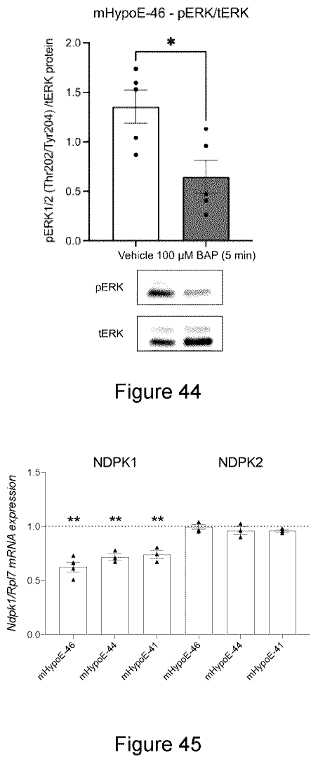

We also assessed the activity of another key signal transduction cascade, the MAPK ERK1/2 pathway. mHypoE-46 neurons were treated with 100 μM BAP and vehicle control for 5 minutes followed by protein isolation and Western blot analysis. The phosphorylation level of ERK1/2, one of the MAPK family members, was assessed. ERK1/2 phosphorylation at Thr202/Tyr204 was significantly decreased by BAP at 5 minutes (see FIG. 44).

BAP Represses the Expression of a Modulator of Cellular ATP Levels Called Nucleoside Diphosphate Kinase (Nadpk1) But Not Nadpk2 in the mHypoE-46 Hypothalamic Cell Line.

We also assessed the expression of a key protein that could potentially be the mammalian homologue of one of the endogenous receptors for BAP in plants called histidine kinase. In mammalian cells nucleoside diphosphate kinase, NADPK1 and NAPPK2, have been postulated to be homologous to the plant histidine kinases. mHypoE-46 neurons were treated with 100 μM BAP and vehicle control for 16 h followed by RNA isolation and qRT-PCR. The expression level of Nadpk1 was significantly decreased, while Nadpk2 levels were not changed (see FIG. 45). The protein activity of NADPKs will be studied upon BAP exposure.

BAP Increases the PI3K and Akt Signal Transduction Pathway in the mHypoE-46 Hypothalamic Cell Line.

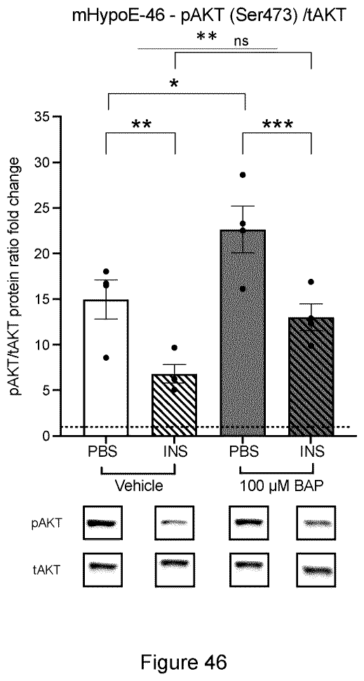

In order to determine if insulin signaling and sensitivity may be affected by BAP, we assessed the activity and expression of the key signal transduction cascade activated by insulin, the PI3K/Akt pathway. mHypoE-46 neurons were co-treated with 100 μM insulin or vehicle control and 100 μM BAP and vehicle control for 24 hours. Insulin was used to induce cellular insulin resistance, as previously reported in our laboratory (25). The neurons were then serum starved for 1 hour in low glucose DMEM and re-exposed to 100 nM insulin for 15 minutes followed by protein isolation and Western blot analysis. The phosphorylation level of Akt (PKB), the downstream kinase in the PI3K/Akt signal transduction pathway was assessed. Akt phosphorylation at Ser473 was significantly increased by BAP upon 15 minutes of insulin exposure in neurons pre-treated with vehicle alone (see FIG. 46). However, this induction of Akt phosphorylation by BAP was not observed in neurons pre-treated with 100 μM insulin for 24 hours (i.e., insulin-induced cellular insulin resistance).

Further, the gene expression of PI3K subunit (Pik3r1) was also significantly increased at 4 and 16 h by BAP (see FIG. 42A and 42B). Taken together, it appears that the PI3K/Akt signal transduction cascade is enhanced by BAP, thus implying that insulin sensitivity would be increased in the neurons.

BAP Increases Insulin Sensitivity in the mHypoE-46 Hypothalamic Cell Line.

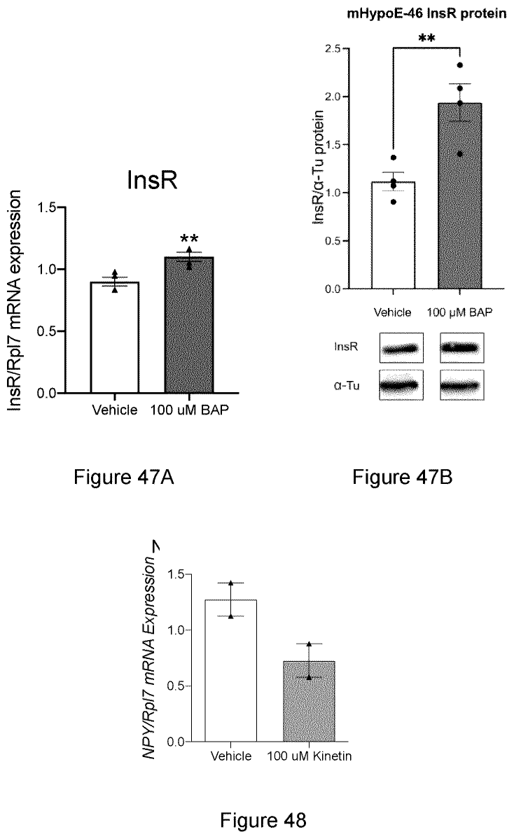

In the animal studies, we found that the ITT and GTT results suggest that BAP may in part reduce body weight by improving glucose homeostasis. The mHypoE-46 neurons were exposed to 100 mM BAP for 16 h followed by RNA isolation. The expression of the insulin receptor subunit beta (Insrb) was increased at 16 h by qRT-PCR when exposed to BAP (see FIG. 47A). Additionally, to assess the effect of BAP on insulin signaling under cellular insulin resistance condition, the neurons were co-treated with 100 μM insulin or PBS and 100 μM BAP or DMSO for 24 hours followed by protein isolation.

Western blot analysis was used to assess for levels of the insulin receptor subunit beta (InsRb). We find that the basal levels of InsRb are significantly increased upon BAP exposure again indicating an enhanced insulin sensitivity (see FIG. 47B).

A Cytokinin Related to BAP Called Kinetin Also Decreases Npy Expression in the mHypoE-46 Hypothalamic Cell Line.

The adenine-derived cytokinins, including kinetin, zeatin, and 6-benzylaminopurine, have similar structures due to their purine backbone. We therefore exposed the Npy-expressing line mHypoE-46 to 100 μM kinetin for 16 h followed by RNA isolation and qRT-PCR analysis of Npy gene expression.

We find that kinetin exposure results in an approximate 50% repression of Npy transcripts (see FIG. 48). However, with an n=2 these experiments will need to be replicated. This suggests that the effect of kinetin in the mHypoE-46 neurons is potentially similar to that of BAP. These studies will be replicated and expanded to determine mechanisms of action of this related adenine-derived cytokinin.

### Discussion

The obesity pandemic continues to plague our society and growing worldwide. The most currently available pharmaceutical options to treat obesity are costly, often ineffective, and have undesirable side effects. Furthermore, most of the choices are only able to achieve sufficient weight loss in combination with lifestyle changes. This has led to only 1-2% of obese patients receiving any form of pharmaceutical intervention. We have identified a future potential obesity therapeutic in BAP. Herein, we have demonstrated the ability of BAP to reduce body weight in CD-1 mice via two different oral administration methods, while on either a 60% HFD or sucrose-matched control diet. The strongest weight loss effect was observed in male CD-1 mice on a 60% HFD. We identify a reduction in food intake as a potential mechanism for the weight loss effects of BAP observed in CD-1 mice. Overall, our results demonstrate the weight loss potential of BAP and its appetite supressing effects.

The strongest weight loss effects of BAP are observed in the male CD-1 mice administered BAP in their drinking water while on a 60% HFD. The peak weight loss seen in this group is greater than that observed in the male CD-1 mice administered a higher dosage of BAP via honey emulsion feeding. This is likely the result of the reduction in water intake observed in the mice provided BAP via their drinking water, which is not evident in the group fed BAP via a honey emulsion. This is not to say that the strong weight loss effects observed in this group are entirely caused by a reduction in water intake, as the control mice drink less water after day 9 of exposure, but they do not experience weight loss. The male mice receiving the BAP emulsion have reduced body weight without any changes in water intake. In addition to this, the female CD-1 mice on the control diet receiving BAP also drink less water, but do not have a similar level of weight loss. Overall, the ability of BAP to reduce weight in CD-1 mice while they are still consuming a 60% HFD is quite remarkable. This would make BAP a desirable alternative compared to present pharmaceutical options that also require their users to adapt lifestyle changes, which is often difficult to maintain.

BAP is able to decrease body weight by reducing food intake. This is observed in the male CD-1 mice fed BAP emulsion daily as we detect an immediate reduction in food intake after only one day of exposure. This reduction in food intake continued throughout the exposure period. However, the decrease in weight seen in the female CD-1 mice is not reflected in an overall decrease in food intake and may be due to changes in metabolic processes instead. This is consistent with the previous EPA study (attached) that implied male rats fed BAP had a reduction in weight and food intake (experimental design and data not shown). Food intake was not measured in the male or female CD-1 mice provided BAP via their drinking water, and as such we are unable to verify that a reduction in food intake is the cause of the weight loss observed in the first set of experiments. However, it appears from our data and the EPA report that BAP acts as an appetite suppressant.

The BAP-mediated weight loss observed in our mouse experiments likely involves the hypothalamus, a key regulator of energy homeostasis. The effects of BAP on hypothalamic feeding neuropeptide expression in the male CD-1 mice fed BAP at 2 hr prior to sacrifice do not reflect the effects of BAP on food intake that we observe, as we see increases in the expression of orexigenic Npy and Agrp. Similar changes in hypothalamic feeding neuropeptide expression are seen in the male CD-1 mice continuously exposed to BAP via their drinking water for 33 days. A potential explanation is that these are compensatory changes in response to the body weight that has been lost with increased appetite-this is also seen in rebound weight gain after dieting. Future experiments involving a time course in mice with oral BAP exposure or intranasal administration to target the hypothalamus in CD-1 mice may prove informative and address the effects of BAP directly on the hypothalamus. To test this direct effect, we treated both the immortalized hypothalamic neurons and cultured primary hypothalamic neurons with BAP and observed a significant increase in Pomc. This suggests that BAP could induce an anorexigenic response in hypothalamic neurons. Overall, these results suggest the involvement of POMC, but further studies are necessary.

Although Npy and Agrp were unaffected by BAP in our primary culture experiments, we did observe changes in our Npy- and Agrp-expressing neuronal cell models. We observed differential effects on Npy expression depending upon the cell lines, but there was a consistent, although modest, induction of Agrp. This illustrates the differential mechanisms potentially involved in regulating orexigenic neuropeptides in hypothalamic neuronal subpopulations. The induction of Npy by BAP in the mHypoE-41 cell line was not entirely unexpected, as there is heterogeneity within Npy-expressing neuronal subpopulations, and previous studies conducted by our lab on the effects of bisphenol A (BPA), a common endocrine disrupting chemical, on Npy expression showed that BPA induced Npy in the mHypoE-41 cell line, but supressed its expression in the mHypoE-46 line (26). The BAP-mediated mechanisms may differ in individual neuronal models. This may also be due to differing glucose levels in treatments.

The structural similarity of BAP to adenine derivatives, combined with a limited number of studies, has suggested that it primarily acts via P2 purine receptors. The P2 family of purine receptors is made up of two major branches, P2Y, which are G-protein coupled receptors, and P2X, which are ATP-gated cation channels. Our hypothalamic neuronal cell lines have been screened and express 10 different P2 receptors, including 4 of 7 P2Y (Y1, Y2, Y4, Y6) and 6 of 7 P2X (X2, X3, X4, X5, X6, X7) receptors, but all were detected in hypothalamic tissue. Multiple P2Y receptors have been implicated in the hypothalamic control of appetite (27). Nearly all of the cell lines tested have the same P2 receptor expression profiles with exception of two lines that did not respond to BAP with a decrease in Npy levels, the mHypoA-59 line, which is the only line screened thus far that expresses P2X1, but also expresses very high levels of P2Y6 and negligible levels of P2Y1 and much lower levels of P2Y2 compared to the other cell lines, and mHypoE-38 line, which is the only line that expresses P2Y14 and much lower levels of P2X5 compared to the other cell lines (Table 2).

TABLE 3
P2 purine receptor screening.

| | P2Y1 | P2Y2 | P2Y4 | P2Y6 | P2Y12 | P2Y13 | P2Y14 | P2X1 | P2X2 | P2X3 | P2X4 | P2X5 | P2X6 | [symbol] |
|---|---|---|---|---|---|---|---|---|---|---|---|---|---|---|
| mHypoE-46 | 28 | 23 | 33 | 29 | 37 | nil | nil | 36 | 28 | 29 | 23 | 25 | 29 | [symbol] |
| mHypoE-41 | 28 | 22 | 33 | 29 | 37 | nil | nil | 38 | 27 | 30 | 23 | 25 | 29 | [symbol] |
| mHypoE-44 | 28 | 21 | 33 | 28 | nil | nil | nil | 39 | 27 | 29 | 22 | 25 | 27 | [symbol] |
| mHypoA-59 | 35 | 27 | 32 | 17 | nil | nil | nil | 31 | 26 | 28 | 21 | 25 | 27 | [symbol] |
| mHypoE-38 | 26 | 23 | 33 | 31 | nil | nil | 26 | 38 | 25 | 29 | 22 | 32 | 27 | [symbol] |
| mHypoA- | 26 | 21 | 33 | 30 | nil | nil | nil | nil | 26 | 27 | 22 | 24 | 26 | [symbol] |
| Bmal1-WT/F | | | | | | | | | | | | | | |
| Hypothalamus | 27 | 27 | 31 | 27 | 27 | 25 | 26 | 28 | 27 | 25 | 23 | 25 | 24 | [symbol] |

nil = no amplification; N.B. CT values >34 considered below threshold of reliable qRT-PCR levels or no expression. [symbol]
indicates data missing or illegible when filed

Coincidentally these are also the cell lines that do not respond to BAP. The nearly identical P2 receptor expression profiles indicate that the differential effects observed are not the result of a single receptor, but potentially due to differences in the downstream signal transduction pathways involved in their effects. This is particularly of interest with regards to the mHypoA-59 cell line, as even though they express the putative receptors for BAP, they show no changes in feeding neuropeptide expression in response to BAP. Yet, this cell line does respond to ATP, the endogenous agonist for most P2 receptors, and we see increased Npy and Agrp expression with ATP (data not shown; as demonstrated in the other three cell lines in FIG. 19), suggesting that BAP may not be acting entirely via P2 signaling in all hypothalamic neurons.

To assess the potential involvement of P2 signaling in the BAP-mediated effects, we cotreated with ATP or UDP. Cotreatment with ATP was able to block any further induction of Npy mRNA levels by BAP in the mHypoE-41 cell line. This indicates the involvement of P2 signaling in this cell line but does not delineate the exact receptor in the family by which BAP is acting, as ATP is the endogenous preferred ligand for all P2X receptors. ATP also binds a variety of P2Y receptors to varying degrees. A potential receptor of interest is P2Y2, which has the greatest affinity for ATP of the P2Y receptors expressed and its knockout has been reported to increase food intake (28). Future studies knocking down P2Y2 with siRNA and treating with BAP may verify this in the hypothalamic neurons.

In the mHypoE-44 cell line, cotreatment with UDP was able to completely block the BAP-mediated downregulation of Npy, indicating that receptors bound by UDP are involved. Of the P2 receptors expressed, UDP is the endogenous agonist for only P2Y6. Previous studies have demonstrated the involvement of P2Y6 in hypothalamic appetite control, as ICV infusion of UDP increased food intake and knockout of P2Y6 in Agrp-expressing neurons blocked this effect (29). GPR17 is a recently de-orphaned receptor, which has structural similarities to cysteinyl leukotriene and P2Y receptors. The recent studies indicate that GPR17 could bind uracil nucleotides including UDP. Furthermore, GPR17 is involved in the hypothalamic control of food intake, as activation of GPR17 in the hypothalamus increases food intake and this effect was dependent on the presence of FOXO1 in Agrp-expressing neurons (30). Another potential G protein-coupled receptor is the reported adenine receptor (AdeR, considered a PO purinergic receptor), which binds adenine and acts through the adenyl cyclase/PKA/cAMP pathway (31, 32). The AdeR is expressed at high levels in the hypothalamus, thus is another key candidate. Furthermore, we are also considering the P1 purinergic receptors, adenosine receptors A1 and A2a, as potential candidate receptors due to the structural similarities of BAP to the purine moiety.

Further studies into the involvement of P2Y6, GPR17, and AdeR in the BAP induced downregulation of Npy in the mHypoE-44 line may provide us with insight into the mechanisms involved. We have also found that BAP can decrease AMPK phosphorylation in mHypoE-46 neurons, indicating that BAP may in part act through this pathway to achieve its decrease in appetite and ultimately weight loss.

The suppression of Npy by BAP in the mHypoE-46 cell line was unaffected by any co-treatments performed so far, suggesting the potential involvement of a P2 receptor that has yet to be tested or an entirely different mechanism. Of the P2Y receptors expressed in this cell line, the remaining agonists yet to be tested are ADP, which is the agonist for P2Y1, also implicated in the hypothalamic control of food intake in rats, and UTP, which is the agonist for P2Y4 (33). Preliminary experiments using UTP do not change the BAP-mediated repression of NPY in the mHypoE-46 cell line. There are specific antagonists available for all of the P2 receptors, and these should be assessed in the cell lines (34, 35). Signaling via these receptors in the mHypoE-46 may be responsible for the effects and future experiments involving co-treatment with these may prove informative.

With regards to other signal transduction pathways, we present evidence that BAP can inhibit the mTORC1 pathways through a number of components, likely resulting in a significant decrease in NPY, appetitie suppressing effects, and weight loss. The components of this pathway that are increased include sestrin2, TSC2, ULK1, and 4E-BP1. The resulting effect is a repression of mTORC1, an decrease in p70-S6K, a significant decrease in the transcription factors Egr1 and Egr3, resulting in a repression of Npy transcription (23).

BAP was also shown to enhance insulin sensitivity in a number of ways in the animals and the neurons. The ITT and GTT indicated an enhanced glucose sensitivity, whereas in the neurons, there was an increase in PI3K/Akt signaling, as well as an increase in the transcription and protein levels of the insulin receptor. Taken together, these indicate that insulin signaling is enhanced which may be a consequence of the overall weight loss in the animals, but also exhibit cellular changes at the level of the neuron itself. These are promising findings that have consequences in the treatment of type 2 diabetes, since besides the benefits of weight loss, an improvement in glucose homeostasis is critical for overall health.

In plants, cytokinins primarily act intracellularly and have specific transporters. There is little known with regards to the potential intracellular effects of BAP in mammalian cells. Due to the structural similarities of BAP to adenine, it is not beyond the realm of possibility that BAP may interact with components involved in the nucleotide salvage pathway allowing for its uptake. This is evident in previous studies involving Hela cells that demonstrated intake of the riboside variant of BAP and metabolism to BAP (36). Overall, this suggests the possibility of an intracellular mechanism of action for BAP and future studies tracking internalization of labeled BAP may prove useful. Further, other cytokinins will be assessed in mice and neurons, specifically kinetin and zeatin. Our preliminary results indicate that kinetin may also decrease Npy expression (see FIG. 46). Of interest, kinetin fed to rats in normal chow caused a decrease in body weight, as a noticeable but unstudied effect, when assessing the role of kinetin on neurodegeneration in the hippocampus (37).

In conclusion, we report that BAP can reduce food intake and body weight in mice potentially via a mechanism involving feeding neuropeptide expression in the hypothalamus. Overall, our results demonstrate the use of BAP as an appetite suppressant in animals, and the use of this common agricultural supplement for plant growth may help to alleviate the ever-growing obesity pandemic afflicting our modern society (38).

While the present use of BAP as an appetite suppressant in mice, it will be appreciated by those skilled in the art that humans will be affected in the same way insofar as appetite suppression upon ingestion of BAP. Rodents have been used extensively as models of human disease (39). There is ample evidence from the literature that preclinical studies are initially performed in mice before translating the findings to humans for metabolic and other types of therapeutics (40). A good example of this progression of preclinical animal studies to the development of a human therapeutic would be the development of Glp-1 agonists as diabetes and obesity therapeutics (41). The inventors contemplate that these findings in the mice will be translated to humans and result in a potent, cost-effective appetite suppressant that can be used in the general population to control weight gain.

Thus, the present disclosure provides an appetite suppressant administered to a subject comprising N6-benzylaminopurine and derivatives thereof formulated in an administrable form to the subject, and the N6-benzylaminopurine may is present in the administrable form in a concentration range from about 50 to about 2000 milligrams/kilogram of the subject's weight. In another embodiment the N6-benzylaminopurine is present in the administrable form in a concentration range from about 100 to about 500 milligrams/kilogram of the subject's weight. In another embodiment the N6-benzylaminopurine is present in the administrable form in a concentration range from about 150 to about 300 milligrams/kilogram of the subject's weight. The N6-benzylaminopurin is present in the administrable form in a concentration of about 200 milligrams/kilogram of the subject's weight.

In an embodiment the N6-benzylaminopurine is in an ingestible solid tablet form. The N6-benzylaminopurine can be in a powder form encased in an ingestible capsule. The N6-benzylaminopurine can be in the form of a liquid. The liquid may be any in which that N6-benzylaminopurine is soluble. The liquid may include 70% ethanol, a solution of 0.02 M NaOH with 2% erythritol or water.

The liquid can be formulated as an administrable intranasal spray.

The N6-benzylaminopurine can be in the form of an ingestible emulsion. The emulsion may comprise a 50% honey format with about 0.5% carboxymethyl cellulose.

The appetite suppressant N6-benzylaminopurine can be used in combination with any other weight loss compounds.

The N6-benzylaminopurine can be used in combination with any other diabetes medications, including insulin sensitizing compounds.

The N6-benzylaminopurine can be used as a treatment for obesity-related diseases, including diabetes, heart disease, depression, fatty liver disease, diabetes-related comorbidities, and polycycstic ovarian disease.

The N6-benzylaminopurine can be used in combination with any other therapeutic compounds known to increase appetite as a side effect.

The N6-benzylaminopurine can be used as an inhibitor of AMP-kinase.

The N6-benzylaminopurine may be used as an inhibitor of mTORC1.

The N6-benzylaminopurine may be used as an inhibitor of mTORC1 acting through induction of Sestrin2 and Tuberous Sclerosis Complex 2 (TSC2).

The N6-benzylaminopurine may be used as an inhibitor of neuropeptide Y (NPY).

The N6-benzylaminopurine may be used as an inhibitor of neuropeptide

Y acting through reduction of S6K phosphorylation and/or changes in transcription factors Early Growth Response Proteins 1 and 3 (Egr1 and Egr3).

The N6-benzylaminopurine may be used as an inducer of proopiomelanocortin (POMC).

The N6-benzylaminopurine may be used as an inducer of Akt phosphorylation and PI3 kinase.

The N6-benzylaminopurine may be used as a repressor of ERK1/2 phosphorylation, a member of the mitogen-activated protein (MAP) kinase family.

The N6-benzylaminopurine may be used as a modulator of nucleoside diphosphate kinase (NDPK).

The N6-benzylaminopurine may be used as an inducer of insulin receptor.

The N6-benzylaminopurine may be used to enhance glucose tolerance and improve insulin sensitivity.

The related cytokinin from the same family kinetin is also capable of suppressing Npy expression and may also act as an appetite suppressant.

The subject animal can be any one of humans, human pets and farm animals.

### REFERENCES

1. Belsham DD, Hanchard J, Bolz SS. Hope in Hopeless Times: Gearing Up to Fight the Obesity Pandemic. Endocrinology. 2020; 161 (10).

2. Saxon DR, Iwamoto SJ, Mettenbrink CJ, McCormick E, Arterbum D, Daley MF, et al. Antiobesity Medication Use in 2.2 Million Adults Across Eight Large Health Care Organizations: 2009-2015. Obesity (Silver Spring). 2019;27 (12): 1975-81.

3. Schwartz MW, Woods SC, Porte D, Jr., Seeley RJ, Baskin DG. Central nervous system control of food intake. Nature. 2000;404 (6778): 661-71.

4. Clegg DJ, Gotoh K, Kemp C, Wortman MD, Benoit SC, Brown LM, et al. Consumption of a high-fat diet induces central insulin resistance independent of adiposity. Physiol Behav. 2011;103 (1): 10-6.

5. El-Haschimi K, Pierroz DD, Hileman SM, Bjorbaek C, Flier JS. Two defects contribute to hypothalamic leptin resistance in mice with diet-induced obesity. The Journal of clinical investigation. 2000; 105 (12): 1827-32.

6. Dalvi PS, Nazarians-Armavil A, Tung S, Belsham DD. Immortalized neurons for the study of hypothalamic function. American journal of physiology Regulatory, integrative and comparative physiology. 2011;300 (5): R1030-52.

7. Othman EM, Naseem M, Awad E, Dandekar T, Stopper H. The Plant Hormone Cytokinin Confers Protection against Oxidative Stress in Mammalian Cells. PLOS One. 2016; 11 (12): e0168386.

8. Wanitphakdeedecha R, Meeprathom W, Manuskiatti W. Efficacy and safety of 0.1% kinetin cream in the treatment of photoaging skin. Indian J Dermatol Venereol Leprol. 2015;81 (5): 547.

9. Stojilkovic SS. Purinergic regulation of hypothalamicuitary functions. Trends Endocrinol Metab. 2009;20 (9): 460-8.

10. Froldi G, Gallo U, Ragazzi E, Caparrotta L. 6-Benzylaminopurine: a plant derived cytokinin inducing positive inotropism by P2-purinoceptors. Planta Med. 1999;65 (3): 245-9.

11. Kim S, Lee J, Jung E, Lee J, Huh S, Hwang H, et al. 6-Benzylaminopurine stimulates melanogenesis via CAMP-independent activation of protein kinase A. Arch Dermatol Res. 2009;301 (3): 253-8.

12. Kuster T, Zumkehr B, Hermann C, Theurillat R, Thormann W, Gottstein B, et al. Voluntary ingestion of antiparasitic drugs emulsified in honey represents an alternative to gavage in mice. J Am Assoc Lab Anim Sci. 2012;51 (2): 219-23.

13. Belsham DD, Cai F, Cui H, Smukler SR, Salapatek AM, Shkreta L. Generation of a phenotypic array of hypothalamic neuronal cell models to study complex neuroendocrine disorders. Endocrinology. 2004; 145 (1): 393-400.

14. Belsham DD, Fick LJ, Dalvi PS, Centeno ML, Chalmers JA, Lee PK, et al. Ciliary neurotrophic factor recruitment of glucagon-like peptide-1 mediates neurogenesis, allowing immortalization of adult murine hypothalamic neurons. FASEB journal: official publication of the Federation of American Societies for Experimental Biology. 2009;23 (12): 4256-65.

15. Lieu CV, Loganathan N, Belsham DD. Mechanisms Driving Palmitate-Mediated Neuronal Dysregulation in the Hypothalamus. Cells. 2021;10 (11).

16. Dalvi PS, Chalmers JA, Luo V, Han D-Y, Wellhauser L, Liu Y, et al. High fat induces acute and chronic inflammation in the hypothalamus: effect of high-fat diet, palmitate and TNF-α on appetite-regulating NPY neurons. International journal of obesity (2005). 2017;41:149-58.

17. Tran A, He W, Chen JTC, Wellhauser L, Hopperton KE, Bazinet RP, et al. Palmitate-mediated induction of neuropeptide Y expression occurs through intracellular metabolites and not direct exposure to proinflammatory cytokines. J Neurochem. 2021;159 (3): 574-89.

18. Martins L, Fernandez-Mallo D, Novelle MG, Vazquez MJ, Tena-Sempere M, Nogueiras R, et al. Hypothalamic mTOR signaling mediates the orexigenic action of ghrelin. PloS one. 2012;7 (10): e46923.

19. Mori H, Inoki K, Munzberg H, Opland D, Faouzi M, Villanueva EC, et al. Critical role for hypothalamic mTOR activity in energy balance. Cell metabolism. 2009;9 (4): 362-74.

20. Varela L, Martinez-Sanchez N, Gallego R, Vazquez MJ, Roa J, Gandara M, et al. Hypothalamic mTOR pathway mediates thyroid hormone-induced hyperphagia in hyperthyroidism. J Pathol. 2012;227 (2): 209-22.

21. Gong L, Wang Z, Wang Z, Zhang Z. Sestrin2 as a Potential Target for Regulating Metabolic-Related Diseases. Front Endocrinol (Lausanne). 2021; 12:751020.

22. Cyr NE, Toorie AM, Steger JS, Sochat MM, Hyner S, Perello M, et al. Mechanisms by which the orexigen NPY regulates anorexigenic alpha-MSH and TRH. Am J Physiol Endocrinol Metab. 2013;304 (6): E640-50.

23. Natt D, Barchiesi R, Murad J, Feng J, Nestler EJ, Champagne FA, et al. Perinatal Malnutrition Leads to Sexually Dimorphic Behavioral Responses with Associated Epigenetic Changes in the Mouse Brain. Sci Rep. 2017;7 (1): 11082.

24 Andersson G, Pahlman S, Parrow V, Johansson I, Hammerling U. Activation of the human NPY gene during neuroblastoma cell differentiation: induced transcriptional activities of AP-1 and AP-2. Cell growth & differentiation: the molecular biology journal of the American Association for Cancer Research. 1994;5 (1): 27-36.

25. Mayer CM, Belsham DD. Central insulin signaling is attenuated by long-term insulin exposure via insulin receptor substrate-1 serine phosphorylation, proteasomal degradation, and lysosomal insulin receptor degradation. Endocrinology. 2010;151 (1): 75-84.

26. Loganathan N, Mcllwraith EK, Belsham DD. BPA Differentially Regulates NPY Expression in Hypothalamic Neurons Through a Mechanism Involving Oxidative Stress. Endocrinology. 2020;161 (11).

27. Caruso V, Zuccarini M, Di lorio P, Muhammad I, Ronci M. Metabolic Changes Induced by Purinergic Signaling: Role in Food Intake. Front Pharmacol. 2021; 12:655989.

28. Zhang Y, Ecelbarger CM, Lesniewski LA, Muller CE, Kishore BK. P2Y2 Receptor Promotes High-Fat Diet-Induced Obesity. Front Endocrinol (Lausanne). 2020;11:341.

29. Steculorum SM, Paeger L, Bremser S, Evers N, Hinze Y, Idzko M, et al. Hypothalamic UDP Increases in Obesity and Promotes Feeding via P2Y6-Dependent Activation of AgRP Neurons. Cell. 2015;162 (6): 1404-17.

30. Ren H, Orozco IJ, Su Y, Suyama S, Gutierrez-Juarez R, Horvath TL, et al. FoxO1 target Gpr17 activates AgRP neurons to regulate food intake. Cell. 2012;149 (6): 1314-26.

31. Fukuda T, Kuroda T, Kono M, Hyoguchi M, Tajiri S, Tanaka M, et al. Adenine attenuates the Ca(2+) contraction-signaling pathway via adenine receptor-mediated signaling in rat vascular smooth muscle cells. Naunyn-Schmiedeberg's archives of pharmacology. 2016;389 (9): 999-1007.

32. Thimm D, Knospe M, Abdelrahman A, Moutinho M, Alsdorf BB, von Kugelgen I, et al. Characterization of new G protein-coupled adenine receptors in mouse and hamster. Purinergic Signal. 2013;9 (3): 415-26.

33. Kittner H, Franke H, Harsch JI, El-Ashmawy IM, Seidel B, Krugel U, et al. Enhanced food intake after stimulation of hypothalamic P2Y1 receptors in rats: modulation of feeding behaviour by extracellular nucleotides. Eur J Neurosci. 2006;24 (7): 2049-56.

34. Kennedy C. The P2Y/P2X divide: How it began. Biochem Pharmacol. 2021;187:114408.

35. Muller CE, Baqi Y, Namasivayam V. Agonists and Antagonists for Purinergic Receptors. Methods Mol Biol. 2020;2041:45-64.

36. Aoki MM, Seegobin M, Kisiala A, Noble A, Brunetti C, Emery RJN. Phytohormone metabolism in human cells: Cytokinins are taken up and interconverted in HeLa cell culture. FASEB Bioadv. 2019;1 (5): 320-31.

37. Orr AL, Rutaganira FU, de Roulet D, Huang EJ, Hertz NT, Shokat KM, et al. Long-term oral kinetin does not protect against alpha-synuclein-induced neurodegeneration in rodent models of Parkinson's disease. Neurochem Int. 2017;109:106-16.

38. Atanasov AG, Waltenberger B, Pferschy-Wenzig EM, Linder T, Wawrosch C, Uhrin P, et al. Discovery and resupply of pharmacologically active plant-derived natural products: A review. Biotechnol Adv. 2015;33 (8): 1582-614.

39. Rydell-Tormanen K, Johnson JR. The Applicability of Mouse Models to the Study of Human Disease. Methods Mol Biol. 2019; 1940:3-22.

40. Williams CT. Food and drug administration drug approval process: A history and overview. Nurs Clin N Amer. 2016;51 (1): 1-11.

41. Knudsen LB, Lau J. The Discovery and Development of Liraglutide and Semaglutide. Front Endocrinol (Lausanne). 2019;10:155.

Therefore what is claimed is:

1. An appetite suppressant administered to a subject, comprising:
N6-benzylaminopurine and derivatives thereof formulated in an administrable form to the subject.

2. The appetite suppressant according to claim 1, wherein the N6-benzylaminopurine is present in the administrable form in a concentration range from about 50 to about 2000 milligrams/kilogram of the subject's weight.

3. The appetite suppressant according to claim 1, wherein the N6-benzylaminopurine is present in the administrable form in a concentration range from about 100 to about 500 milligrams/kilogram of the subject's weight.

4. The appetite suppressant according to claim 1, wherein the N6-benzylaminopurine is present in the administrable form in a concentration range from about 150 to about 300 milligrams/kilogram of the subject's weight.

5. The appetite suppressant according to claim 1, wherein the N6-benzylaminopurin is present in the administrable form in a concentration of about 200 milligrams/kilogram of the subject's weight.

6. The appetite suppressant according to any one of claims 1 to 5, wherein the N6-benzylaminopurine is in an ingestible solid tablet form.

7. The appetite suppressant according to any one of claims 1 to 5, wherein the N6-benzylaminopurine is in a powder form encased in an ingestible capsule.

8. The appetite suppressant according to any one of claims 1 to 5, wherein the N6-benzylaminopurine is in the form of a liquid.

9. The appetite suppressant according to claim 8, wherein the liquid is any in which that N6-benzylaminopurine is soluble.

10. The appetite suppressant according to claim 8, wherein the liquid includes 70% ethanol, a solution of 0.02 M NaOH with 2% erythritol or water.

11. The appetite suppressant according to claim 8, wherein the liquid is formulated as an administrable intranasal spray.

12. The appetite suppressant according to any one of claims 1 to 5, wherein the N6-benzylaminopurine is in the form of an ingestible emulsion.

13. The appetite suppressant according to claim 12, wherein the emulsion comprises a 50% honey format with about 0.5% carboxymethyl cellulose.

14. The appetite suppressant according to any one of claims 1 to 13, wherein the N6-benzylaminopurine is used in combination with any other weight loss compounds.

15. The appetite suppressant according to any one of claims 1 to 14, wherein the N6-benzylaminopurine is used in combination with any other diabetes medications, including insulin sensitizing compounds.

16. The appetite suppressant according to any one of claims 1 to 15, wherein the N6-benzylaminopurine is used as a treatment for obesity-related diseases, including diabetes, heart disease, depression, fatty liver disease, diabetes-related comorbidities, and polycycstic ovarian disease.

17. The appetite suppressant according to any one of claims 1 to 16, wherein the N6-benzylaminopurine is used in combination with any other therapeutic compounds known to increase appetite as a side effect.

18. The appetite suppressant according to any one of claims 1 to 17, wherein the subject animal is any one of humans, human pets and farm animals.

19. The use of N6-benzylaminopurine and derivatives thereof as an inhibitor of AMP-kinase.

20. The use of N6-benzylaminopurine and derivatives as an inhibitor of mTORC120.

21. The use of N6-benzylaminopurine according to claim 19, wherein the N6-benzylaminopurine is used as an inhibitor of mTORC1 acting through induction of Sestrin2 and Tuberous Sclerosis Complex 2 (TSC2).

22. The use of N6-benzylaminopurine and derivatives as an inhibitor of neuropeptide Y.

23. The appetite suppressant according to claim 22, wherein the N6-benzylaminopurine is used as an inhibitor of neuropeptide Y (NPY) acting through reduction of S6K phosphorylation and/or changes in transcription factors Early Growth Response Proteins 1 and 3 (Egr1 and Egr3).

24. The use of N6-benzylaminopurine and derivatives as an inducer of proopiomelanocortin (POMC).

25. The use of N6-benzylaminopurine and derivatives as an inducer of Akt phosphorylation and PI3 kinase.

26. The use of N6-benzylaminopurine and derivatives as a repressor of ERK1/2 phosphorylation.

27. The use of N6-benzylaminopurine and derivatives as a modulator of nucleoside diphosphate kinase (NDPK).

28. The use of N6-benzylaminopurine and derivatives as an inducer of insulin receptor.

29. The use of N6-benzylaminopurine and derivatives to enhance glucose tolerance and improve insulin sensitivity.

30. The use of N6-benzylaminopurine and derivatives according to any one of claims 19 to 29, wherein the subject is any one of humans, human pets, farm animals

31. The use of kinetin as an appetite suppressant.
